(19) United States
(12) Patent Application Publication
(10) Pub. No.: US 2025/0101117 A1
(43) Pub. Date: Mar. 27, 2025

(54) METHODS FOR TREATING INFLAMMATORY BOWEL DISEASE

(71) Applicant: Genentech, Inc., South San Francisco, CA (US)

(72) Inventors: Lin Pan, Sunnyvale, CA (US); Horace H. Rhee, San Mateo, CA (US); Fei Tang, Foster City, CA (US)

(21) Appl. No.: 18/820,893

(22) Filed: Aug. 30, 2024

Related U.S. Application Data
(60) Provisional application No. 63/661,390, filed on Jun. 18, 2024.
(60) Provisional application No. 63/600,980, filed on Nov. 20, 2023.
(60) Provisional application No. 63/580,189, filed on Sep. 1, 2023.

(51) Int. Cl. C07K 16/28; A61K 39/00; A61K 45/06

(52) U.S. Cl. CPC C07K 16/2866; A61K 45/06; A61K 2039/505; A61K 2039/545

(57) ABSTRACT

This disclosure relates to methods, uses, and compositions (e.g., articles of manufacture and kits) comprising an anti-OSMRβ antibody, such as vixarelimab, for the treatment of inflammatory gastrointestinal diseases associated with the oncostatin M (OSM) pathway (e.g., inflammatory bowel disease (IBD) (e.g., ulcerative colitis (UC (e.g., moderate to severe UC)) and Crohn's disease (CD)).

### CROSS-REFERENCE TO RELATED APPLICATIONS

This application claims priority to and benefit of U.S. Provisional Patent Application No. 63/580,189, filed Sep. 1, 2023; U.S. Provisional Patent Application No. 63/600,980, filed Nov. 20, 2023; and U.S. Provisional Patent Application No. 63/661,390, filed Jun. 18, 2024, the contents of each of the foregoing applications are hereby incorporated by reference in their entireties.

### FIELD OF THE DISCLOSURE

The present disclosure pertains to the field of treatment of Inflammatory Bowel Disease, such as Ulcerative colitis (UC) using an anti-OSMRβ antibody, such as vixarelimab.

### SEQUENCE LISTING

The instant application contains a Sequence Listing which has been submitted electronically in XML format and is hereby incorporated by reference in its entirety. Said XML copy, created on Aug. 30, 2024, is named 000218-0087-101-SL.xml and is 5,175 bytes in size.

### BACKGROUND OF THE DISCLOSURE

Ulcerative colitis (UC) is a chronic inflammatory condition that affects the rectum and extends proximally to the colon in a diffuse, continuous, superficial pattern. UC is characterized by mucosal ulceration, rectal bleeding, diarrhea, and abdominal pain and may be complicated by severe bloody diarrhea and toxic megacolon, requiring major and sometimes urgent surgery. One-third of patients with UC can also experience extra-intestinal complications affecting the skin, joints, eyes, mouth, liver, and lungs (Ungaro et al. 2017; Kobayashi et al. 2020). Although there are many risk factors associated with the development of UC, the disease fundamentally represents dysregulation of the mucosal immune system in a genetically susceptible individual in response to commensal microbiota and other environmental triggers.

The burden of UC is rising, with increasing worldwide incidence and prevalence over time (Unagaro et al. 2017). The highest incidence rates have been reported in Europe (0.6 to 24.3 per 100,000), North America (8.8 to 23.1 per 100,000), and Oceania (7.3 to 17.41 per 100,000) (Du and Ha 2020). While the incidence of UC is stabilizing in Western countries, the prevalence continues to rise, and incidence is increasing in many newly industrialized countries in South America, Asia, Africa, and the Middle East (Ng et al. 2017; Du and Ha 2020; Mak et al. 2020). This rise may be due in part to better detection and diagnosis as well as environmental factors such as improved hygiene and a Western diet. The disease can affect any age group, but onset peaks between the ages of 15 and 35 years.

Current pharmacologic management of UC includes conventional therapies: anti-inflammatory drugs (corticosteroids and aminosalicylates such as 5-aminosalicylic acid [5-ASA]), immunosuppressants (such as azathioprine [AZA], 6-mercaptopurine [6-MP], and methotrexate [MTX]); and advanced therapies: tumor necrosis factor (TNF) inhibitors, anti-integrin agents (e.g., vedolizumab), anti-interleukin agents (e.g., ustekinumab), Janus kinase (JAK) inhibitors (e.g., tofacitinib, upadacitinib, filgotinib), and sphingosine-1-phosphate receptor modulators (e.g., ozanimod) (Unagaro et al. 2017; Aslam et al. 2022). Current treatment strategies are focused on disease modification via induction of mucosal healing, decreased dependence on corticosteroids, and reduction in the probability of progression to surgery, without significant compromise of immune competence (Danese et al. 2020; Cai et al. 2021; Le Berre et al. 2022).

Despite the availability of advanced therapies, a therapeutic ceiling remains in patients with moderate to severe UC, with remission rates of only 20%-30% in induction trials (Alsoud et al. 2021). Recent estimates of remission rates from an international survey are 37%-55% with current treatments; however, 22%-29% of patients have experienced a loss of response to current medications (i.e., anti-TNF therapy, anti-integrin, JAK inhibitor, or anti-IL-12/23) (Rubin et al. 2021), highlighting the need for more durable treatment options for patients with UC. Additionally, available advanced therapies have been associated with serious infections, infusion reactions, cardiovascular events, thrombosis, and malignancies (Gordon et al. 2015; XELJANZ® [tofacitinib] U.S. Prescribing Information; RINVOQ® [upadacitinib] U.S. Prescribing Information; STELARA® [ustekinumab] U.S. Prescribing Information; HUMIRA® [adalimumab] U.S. Prescribing Information). Therefore, there remains an unmet need for additional safe UC treatments that are both well-tolerated and have durable efficacy.

Vixarelimab is a first-in-class, fully human, monoclonal antibody that targets oncostatin M receptor-beta (OSMRB). OMR is a cytokine receptor subunit that heterodimerizes with interleukin (IL)-31 receptor-alpha or glycoprotein 130 (gp130) to form two distinct cytokine receptors, IL-31 and oncostatin M (OSM) receptors, which mediate signaling of the cytokines IL-31 and OSM, respectively.

In light of the limitations of currently available therapies, use of vixarelimab is described herein as a novel therapeutic agent to achieve clinical remission in patients with moderate to severe UC, some of whom have demonstrated inadequate response to, loss of response to, or intolerance to prior conventional or advanced therapy.

### SUMMARY OF THE DISCLOSURE

In one aspect, a method for treating a gastrointestinal inflammatory condition associated with the oncostatin M (OSM) signaling pathway in a subject in need thereof is provided. In some embodiments, the gastrointestinal inflammatory condition is an inflammatory bowel disease (IBD). In other embodiments, the IBD is ulcerative colitis (UC) or Crohn's Disease (CD). In preferred embodiments, the IBD is UC. In some embodiments, the UC is moderate to severe UC. In some embodiments, the UC is active moderate to severe UC.

In some embodiments of any of the above aspects, the subject has been diagnosed with or determined to suffer from one or more inflammatory bowel diseases (IBDs). In other embodiments, the IBD is ulcerative colitis (CD) or Crohn's disease (CD). In some embodiments, the UC is moderate to severe UC. In some embodiments, the UC is active moderate to severe UC.

In some embodiments, the anti-OSMRβ antibody comprises a heavy chain variable domain (VH) comprising SEQ ID NO: 3 and a light chain variable domain (VL) comprising SEQ ID NO: 4. In other embodiments, the anti-OSMRβ antibody comprises a heavy chain (HC) comprising SEQ ID NO: 1 and a light chain (LC) comprising SEQ ID NO: 2. In preferred embodiments, the anti-OSMRβ antibody is vixarelimab. In other embodiments, the anti-OSMRβ antibody is a variant of vixarelimab that inhibits activation of the OSMRβ pathway by oncostatin M (OSM) and interleukin-31 (IL-31) as determined in an in vitro cellular assay as described in U.S. Pat. Appn. Pub. No. 2017/0008958. In some embodiments, the anti-OSMRβ antibody is an antibody that competes with vixarelimab for binding to the OSMRβ and that inhibits activation of the OSMRβ pathway by oncostatin M (OSM) and interleukin-31 (IL-31).

In some embodiments, the method comprises administering to the subject 360 mg, 540 mg, or 720 mg of the anti-OSMRβ antibody once every 1 week, once every 2 weeks, once every 3 weeks, once every 4 weeks, or once every month. In preferred embodiments, the method comprises administering to the subject 720 mg of the anti-OSMRβ antibody about once every 2 weeks or about once every 4 weeks.

In some embodiments, the method comprises administering to the subject 720 mg of the anti-OSMRβ antibody (e.g., vixarelimab) once per week for 1 to 12 weeks, then administering to the subject 720 mg of the anti-OSMRβ antibody (e.g., vixarelimab) once every 2 weeks for at least 2, 4, 6, 8, 10, or 12 weeks.

In some embodiments, the method comprises administering to the subject 720 mg of the anti-OSMRβ antibody (e.g., vixarelimab) once per week for 1 to 12 weeks prior to administering to the subject 720 mg of the anti-OSMRβ antibody (e.g., vixarelimab) once every 4 weeks for at least 4, 8, 12, 16, 24, 28, 32, or 48 weeks. In some embodiments, the method comprises administering to the subject 720 mg of the anti-OSMRβ antibody (e.g., vixarelimab) once per week for 1 to 12 weeks prior to administering to the subject 720 mg of the anti-OSMRβ antibody (e.g., vixarelimab) once every 2 weeks for at least 4, 8, 12, 16, 24, 28, 32, or 48 weeks. In some embodiments, the method comprises administering to the subject 720 mg of the anti-OSMRβ antibody (e.g., vixarelimab) administered at weeks 0, 1, 2, and every 2 weeks (Q2W) thereafter. In some embodiments, the method comprises administering to the subject 720 mg of the anti-OSMRβ antibody (e.g., vixarelimab) administered at weeks 0, 1, 4, and every 4 weeks (Q4W) thereafter. In some embodiments, the method comprises administering to the subject 720 mg of the anti-OSMRβ antibody (e.g., vixarelimab) administered at Q2W.

In some embodiments, the method comprises administering to the subject 720 mg of the anti-OSMRβ antibody (e.g., vixarelimab) once per week for 1 week, 2 weeks, or 3 weeks prior to administering to the subject 720 mg of the anti-OSMRβ antibody (e.g., vixarelimab) antibody once every 2 weeks for at least 4, 8, 12, 16, 24, 28, 32, or 48 weeks.

In some embodiments, the method comprises administering to the subject 720 mg of the anti-OSMRβ antibody (e.g., vixarelimab) once per week for 1 week, 2 weeks, or 3 weeks prior to administering to the subject 720 mg of the anti-OSMRβ antibody (e.g., vixarelimab) once every 4 weeks for at least 4, 8, 12, 16, 24, 28, 32 or 48 weeks.

In some embodiments, the anti-OSMRβ antibody (e.g., vixarelimab) is administered subcutaneously. In other embodiments, the anti-OSMRβ antibody (e.g., vixarelimab) is administered intravenously.

In some embodiments of any of the above aspects, the therapeutically effective dose is about 720 mg of the anti-OSMRβ antibody (e.g., vixarelimab) for at least 12 weeks, wherein the anti-OSMRβ antibody (e.g., vixarelimab) is administered at weeks 0, 1, 2, and every 2 weeks (Q2W) thereafter. In some embodiments of any of the above aspects, the therapeutically effective dose is about 720 mg of the anti-OSMRβ antibody (e.g., vixarelimab) for at least 12 weeks, wherein the anti-OSMRβ antibody (e.g., vixarelimab) is administered at weeks 0, 1, 4, and every 4 weeks (Q4W) thereafter. In some embodiments of any of the above aspects, the anti-OSMRβ antibody (e.g., vixarelimab) is administered for 48 weeks.

In some embodiments of any of the above aspects, the anti-OSMRβ antibody (e.g., vixarelimab) is administered subcutaneously. In some embodiments of any of the above aspects, the UC is active. In some embodiments of any of the above aspects, the UC is active moderate to severe UC.

In some embodiments of any of the above aspects, the administering with a therapeutically effective dose of the anti-OSMRβ antibody (e.g., vixarelimab) for at least 10, 11, or 12 weeks results in clinical remission in the subject. In some embodiments of any of the above aspects, the administering with a therapeutically effective dose of the anti-OSMRβ antibody (e.g., vixarelimab) for at least 10 weeks results in clinical remission in the subject. In some embodiments of any of the above aspects, the administering with a therapeutically effective dose of the anti-OSMRβ antibody (e.g., vixarelimab) for at least 11 weeks results in clinical remission in the subject. In some embodiments of any of the above aspects, the administering with a therapeutically effective dose of the anti-OSMRβ antibody (e.g., vixarelimab) for at least 12 weeks results in clinical remission in the subject. In some embodiments, the therapeutically effective dose is administered for at least 48 weeks. In some embodiments, the clinical remission is sustained at 48 weeks.

In some embodiments, the clinical remission in the subject is demonstrated by a modified Mayo Score (mMS) of ≤2. In some embodiments, the clinical remission in the subject is demonstrated by a stool frequency subscore≤1. In some embodiments, the clinical remission in the subject is demonstrated by a rectal bleeding score=0. In some embodiments, the clinical remission in the subject is demonstrated by an endoscope subscore≤1, wherein the endoscopy subscore of 1 is modified to exclude friability. In other embodiments, the clinical remission in the subject is demonstrated by one or more of a mMS of ≤2, a stool frequency subscore≤1, a rectal bleeding score=0, and an endoscope subscore≤1, wherein the endoscopy subscore of 1 is modified to exclude friability. In other embodiments, the clinical remission in the subject is demonstrated by two or more of a mMS of ≤2, a stool frequency subscore≤1, a rectal bleeding score=0, and an endoscope subscore≤1, wherein the endoscopy subscore of 1 is modified to exclude friability. In other embodiments, the clinical remission in the subject is demonstrated by three or more of a mMS of ≤2, a stool frequency subscore≤1, a rectal bleeding score=0, and an endoscope subscore≤1, wherein the endoscopy subscore of 1 is modified to exclude friability. In still other embodiments, the clinical remission in the subject is demonstrated by a mMS of ≤2, a stool frequency subscore≤1, a rectal bleeding score=0, and an endoscope subscore≤1, wherein the endoscopy subscore of 1 is modified to exclude friability.

In some embodiments of any of the above aspects, the administering with a therapeutically effective dose of an anti-OSMRβ antibody (e.g., vixarelimab) for at least 10, 11, or 12 weeks results in a clinical response in the subject. In some embodiments of any of the above aspects, the administering with a therapeutically effective dose of the anti-OSMRβ antibody (e.g., vixarelimab) for at least 10 weeks results in a clinical response in the subject. In some embodiments of any of the above aspects, the administering with a therapeutically effective dose of the anti-OSMRβ antibody (e.g., vixarelimab) for at least 11 weeks results in a clinical response in the subject. In some embodiments of any of the above aspects, the administering with a therapeutically effective dose of the anti-OSMRβ antibody (e.g., vixarelimab) for at least 12 weeks results in a clinical response in the subject. In some embodiments, the therapeutically effective dose is administered for at least 48 weeks. In some embodiments, the clinical response is sustained at 48 weeks.

The clinical response in the subject is demonstrated by the subject experiencing a decrease from baseline in the mMS of >2 and ≥30% reduction from baseline. In some embodiments, the clinical response in the subject is demonstrated by a decrease in rectal bleeding subscore of >1 or absolute rectal bleeding subscore of ≤1. In some embodiments, the clinical response in the subject is demonstrated by an endoscopic improvement at week 12 wherein endoscopic improvement is defined as a Mayo endoscopy subscore of ≤1 (score of 1 modified to exclude friability). In some embodiments, the clinical response in the subject is demonstrated by an endoscopic remission wherein endoscopic remission is defined as a Mayo endoscopy subscore of 0. In some embodiments, the clinical response in the subject is demonstrated by one or more of the subject experiencing a decrease from baseline in the mMS of ≥2 and ≥30% reduction from baseline, a decrease in rectal bleeding subscore of ≥1 or absolute rectal bleeding subscore of ≤1, an endoscopic improvement at week 12 wherein endoscopic improvement is defined as a Mayo endoscopy subscore of ≤1 (score of 1 modified to exclude friability), and/or an endoscopic remission wherein endoscopic remission is defined as a Mayo endoscopy subscore of 0. In some embodiments, the clinical response in the subject is demonstrated by two or more of the subject experiencing a decrease from baseline in the mMS of ≥2 and ≥30% reduction from baseline, a decrease in rectal bleeding subscore of ≥1 or absolute rectal bleeding subscore of ≤1, an endoscopic improvement at week 12 wherein endoscopic improvement is defined as a Mayo endoscopy subscore of ≤1 (score of 1 modified to exclude friability), and/or an endoscopic remission wherein endoscopic remission is defined as a Mayo endoscopy subscore of 0. In some embodiments, the clinical response in the subject is demonstrated by three or more of the subject experiencing a decrease from baseline in the mMS of ≥2 and ≥30% reduction from baseline, a decrease in rectal bleeding subscore of ≥1 or absolute rectal bleeding subscore of ≤1, an endoscopic improvement at week 12 wherein endoscopic improvement is defined as a Mayo endoscopy subscore of ≤1 (score of 1 modified to exclude friability), and/or an endoscopic remission wherein endoscopic remission is defined as a Mayo endoscopy subscore of 0. In other embodiments, the clinical response in the subject is demonstrated by the subject experiencing a decrease from baseline in the mMS of ≥2 and ≥30% reduction from baseline, a decrease in rectal bleeding subscore of ≥1 or absolute rectal bleeding subscore of ≤1, an endoscopic improvement at week 12 wherein endoscopic improvement is defined as a Mayo endoscopy subscore of ≤1 (score of 1 modified to exclude friability), and an endoscopic remission wherein endoscopic remission is defined as a Mayo endoscopy subscore of 0.

In some embodiments, the clinical response is further demonstrated by the subject experiencing an endoscopic improvement at week 12 wherein endoscopic improvement is defined as a Mayo endoscopy subscore of ≤1 (score of 1 modified to exclude friability), and/or an endoscopic remission wherein endoscopic remission is defined as a Mayo endoscopy subscore of 0. In some embodiments, the clinical response is demonstrated by endoscopic improvement at week 12. In some embodiments, the clinical response is demonstrated by endoscopic remission at week 12. In some embodiments, the therapeutically effective dose is administered for at least 48 weeks. In some embodiments, the endoscopic improvement is sustained at week 48 of treatment. In some embodiments, the endoscopic remission is sustained at week 48 of treatment.

In some embodiments, the method results in improved signs or improved symptoms in the subject. In some embodiments, the improved signs or improved symptoms comprise an improvement in ulcerative colitis bowel movement signs and symptoms defined as the proportion of subjects with a greater than or equal to 6-point decrease in the UC-PRO/SS bowel domain score. In some embodiments, the improved signs or improved symptoms comprise a change from baseline in UC functional signs and symptoms as assessed by IC-PRO/SS score. In some embodiments, the improved signs or improved symptoms comprise an improvement in ulcerative colitis functional symptoms as defined by proportion of subjects with a greater than or equal to 2-point increase in the UC-PRO/SS functional domain score. In some embodiments, the improved signs or improved symptoms are assessed at week 12 and week 48. In some embodiments, the treatment with a therapeutically effective dose of the anti-OSMRβ antibody (e.g., vixarelimab) for at least 48 weeks results in a sustained remission.

In some embodiments, the subject has inadequate response to, loss of response to, or intolerance to up to two prior classes of approved ulcerative colitis advanced therapies and/or inadequate response to, loss of response to, or intolerance to prior conventional ulcerative colitis therapies and/or inadequate response to, loss of response to, or intolerance to prior immunosuppressant treatment. In some embodiments, the subject has inadequate response to, loss of response to, or intolerance to up to two prior classes of approved ulcerative colitis advanced therapies. In some embodiments, the subject has inadequate response to, loss of response to, or intolerance to prior conventional ulcerative colitis therapies. In some embodiments, the subject has inadequate response to, loss of response to, or intolerance to prior immunosuppressant treatment.

In some embodiments of any of the above aspects, the subject is receiving anti-tumor necrosis factor (anti-TNF) therapy.

In some embodiments of any of the above aspects, the subject has not been diagnosed with or is not experiencing a lung fibrotic disease or disorder.

In some embodiments of any of the above aspects, the subject has not been diagnosed with or is not experiencing a fibrotic skin disease such as PN or AD.

In some embodiments of any of the above aspects, the anti-OSMRβ antibody (e.g., vixarelimab) is administered in combination with a second therapeutic agent. In other embodiments, the second therapeutic agent is a therapeutic agent indicated for an inflammatory bowel disease. In some embodiments, the second therapeutic agent is an anti-TNFα antibody. In some embodiments, the anti-TNFα antibody is infliximab, adalimumab, golimumab, or a biosimilar thereof. In some embodiments, the second therapeutic agent is an anti-inflammatory agent. In some embodiments, the anti-inflammatory is 5-aminosalicylic acid or a corticosteroid. In some embodiments, the second therapeutic agent is an immunosuppressant. In some embodiments, the immunosuppressant is azathioprine, methotrexate, or 6-mercaptopurine. In some embodiments, the second therapeutic agent is an anti-IL-6 antibody or an anti-IL-6 receptor antibody. In some embodiments, the anti-OSMRβ antibody is administered before, during, or after administration with the second therapeutic agent. In some embodiments, the anti-OSMRβ antibody is administered to the subject after the subject has been treated with the second therapeutic agent for at least 1 week, 1 month, 6 months, 1 year, 3 years, or 5 years.

### BRIEF DESCRIPTION OF THE DRAWINGS

FIGS. 1A-1C provide the PK/PD following a single IV dose of vixarelimab to determine efficacious concentration (C_{eff}) in non-human primate itch models. Specifically, scratching events were plotted against vixarelimab serum concentration at doses of 1 mg/kg (FIG. 1A), 3 mg/kg (FIG. 1B), and 10 mg/kg (FIG. 1C). Vixarelimab was administered intravenously on day 1; 6 animals were treated per dose group. Scratching events are calculated as post IL-31 challenge minus pre-IL-31 challenge events. The lower limit of quantification=0.04 μg/mL and the efficacious concentration was defined via PK/PD correlation is 5 to 8.5 μg/mL.

FIG. 2 demonstrates dose-dependent elimination of vixarelimab via a target-mediated drug disposition. Specifically, a sustained efficacy lasting 6-8 weeks following a single IV dose of 7.5 mg/kg in patients with atopic dermatitis supported C_{eff} of 5-8 μg/mL identified in a non-human primate challenge model. Doses tested include 0.3 mg/kg intravenously (IV), green line; 1.5 mg/kg IV, brown line; 1.5 mg/kg subcutaneously (SC), blue line; and 7.5 mg/kg IV, maroon line. The X-axis is time after dose in weeks, and the Y-axis is concentration in μg/mL.

FIG. 3 demonstrates that a single dose of 7.5 mg/kg vixarelimab can reduce Pruritus worst itch versus placebo. Specifically, vixarelimab recipients experienced a greater improvement in Worst Itch Numeric Rating Scale (WI-NRS) compared with placebo. The improvement in WI-NRS began as early as week 1 (−33% vs −18.5%) increased throughout the monotherapy period (week 4, −43.8% vs −21.2%) and persisted in the adjunctive period with coadministration of topical corticosteroids (TCS; week 6, −51.1% vs −26.3%). Vixarelimab was given during a 28-day monotherapy period plus a 30-day follow up period with TCS as adjunctive therapy. TCS was not allowed during the first four weeks of the study but was permitted beginning after week four (as indicated by the vertical blue dashed line). The solid purple lower line is the 7.5 mg/kg vixarelimab treatment group, and the black dashed upper line is the placebo group. The X-axis is time in weeks, and the Y-axis is Mean (±Standard Error of the Mean (SEM)) % Change from Baseline in WI-NRS.

FIG. 4 demonstrates the simulated median concentration of the vixarelimab PK profiles after a flat dose of 720 mg every two weeks (Q2W), 540 mg Q2W, 360 mg Q2W, and 720 mg every four weeks (Q4W).

FIG. 5 demonstrates the percent of patients above target concentrations with C_{min,ss} with target serum vixarelimab concentrations (μg/mL) after a flat dose of 720 mg every two weeks (Q2W), 540 mg Q2W, 360 mg Q2W, and 720 mg every four weeks (Q4W).

FIG. 6 provides details regarding restrictions for use of concomitant therapies by subjects who participate in a phase 2 study described herein based on US standards.

FIG. 7 describes the Mayo scoring system used to assess subjects who participate in a phase 2 study as described herein.

FIG. 8 provides details regarding restrictions for use of concomitant therapies by subjects who participate in a phase 2 study described herein based on European standards.

### DETAILED DESCRIPTION

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA, and related fields as are within the skill of the art. These techniques are fully explained in the literature.

The term “herein” means the entire application.

It should be understood that any of the embodiments described herein, including those described under different aspects of the disclosure and different parts of the specification (including embodiments described only in the Examples) can be combined with one or more other embodiments disclosed herein, unless explicitly disclaimed or improper.

Any publications, patents and published patent applications referred to in this application are specifically incorporated by reference herein. In case of conflict, the present specification, including its specific definitions, will control.

Throughout this specification, the word “comprise,” or variations such as “comprises” or “comprising,” which is synonymous with “including,” “containing,” or “characterized by,” is inclusive or open-ended and does not exclude additional, unrecited elements or method steps.

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “consisting of” excludes any element, step, or ingredient not specifically recited.

The term “consisting essentially of” limits the scope of a disclosure to the specified materials or steps and those that do not materially affect the basic and novel characteristic(s) of the disclosure.

Any example(s) following the term “e.g.” or “for example” is not meant to be exhaustive or limiting.

The articles “a,” “an” and “the” are used herein to refer to one or to more than one (i.e., to at least one) of the grammatical object of the article. By way of example, “an element” means one element or more than one element.

Exemplary methods and materials are described herein, although methods and materials similar or equivalent to those described herein can also be used in the practice or testing of the present application. The materials, methods, and examples are illustrative only and not intended to be limiting.

### Definitions

The following terms, unless otherwise indicated, shall be understood to have the following meanings:

As used herein, the term “approximately” or “about,” as applied to one or more values of interest, refers to a value that is similar to a stated reference value. In certain embodiments, the term “approximately” or “about” refers to a range of values that fall within 10% or less in either direction (greater than or less than) of the stated reference value unless otherwise stated or otherwise evident from the context (e.g., where such number would exceed 100% of a possible value). Reference to “about” a value or parameter herein includes (and describes) embodiments that are directed to that value or parameter per se. For example, description referring to “about X” includes description of “X.” Numeric ranges are inclusive of the numbers defining the range.

The terms “subject,” “individual,” and “patient” are used interchangeably herein and refer to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual, subject or patient is a human.

The term “OSM,” as used herein, refers to oncostatin M. The term “IL-31,” as used herein, refers to interleukin-31. Both OSM and IL-31 are well-known cytokines which are members of the IL-6 superfamily. The term “OSMR,” as used herein, refers to the oncostatin M receptor and is also referred to herein as “OSMRB” or “OSMR Type II.” OSM is a member of the type I cytokine receptor family. OSMRβ heterodimerizes with glycoprotein 130 (also known herein as gp130) to form the type II OSMR which transduces OSM-induced signaling events. OSMRβ also heterodimerizes with IL-31 receptor A (IL31RA) to form the IL-31 receptor which transduces IL-31-induced signaling events. An exemplary human OSMRβ amino acid sequence is provided in GenBank Accession No. NP 003990.

As used herein, the term “vixarelimab” refers to a monoclonal antibody that targets oncostatin M receptor beta (OSMRB), which mediates signaling of interleukin-31 (IL-31) and oncostatin M (OSM) protein having the heavy and light chain amino acid sequences listed in the International Nonproprietary Names for Pharmaceutical Substances (INN) List 85 (WHO Drug Information, Vol. 35, No. 1, 2021, pp. 228-229). Vixarelimab is also known in some non-patent publications as “KPL 716.”

“Vixarelimab or a variant thereof” is used herein to include vixarelimab in addition to an amino acid variant of the vixarelimab heavy and/or light chain sequences that is shown to inhibit OSMR and IL-31 signaling to an extent similar to that of vixarelimab as determined in an in vitro cellular assay known in the art (e.g., US Pat. Appn. Pub. No. 2017/0008958).

As used herein, the term “biologics” refers to products derived from living organisms that can be used for the methods and uses described herein. Examples of biologics include etanercept; an antibody to tumor necrosis factor alpha, e.g., infliximab, adalimumab or certolizumab; an antibody to IL-12 and IL-23, e.g., ustekinumab; vedolizumab; etrolizumab, and natalizumab.

As used herein, “steroids” refers to organic compounds with a characteristic molecular structure containing four rings of carbon atoms (three six-membered and one five). Examples of a steroid include corticosteroids or glucocorticosteroids. Examples of a corticosteroid include prednisone and hydrocortisone or methylprednisolone, or a second generation corticosteroid, e.g., budesonide or azathioprine; e.g., in forms like a hydrocortisone enema or a hydrocortisone foam.

The terms “inflammatory bowel disorder,” “inflammatory bowel disease,” or “IBD,” as used interchangeably herein, are used herein in the broadest sense and include all diseases and pathological conditions the pathogenesis of which involves recurrent inflammation in the intestine, including small intestine and colon. IBD includes, e.g., ulcerative colitis (UC) and Crohn's disease (CD). IBD is not limited to UC and CD. The manifestations of the disease include but are not limited to inflammation and a decrease in epithelial integrity in the intestine.

The terms “intestine” or “gut” as used interchangeably herein broadly encompasses the small intestine and large intestine.

The term “ulcer” is a site of damage to the skin or mucous membrane that is often characterized by the formation of pus, death of tissue, and is frequently accompanied by an inflammatory reaction.

By “reduce” or “inhibit” is meant the ability to cause an overall decrease preferably of 20% or greater, more preferably of 50% or greater, and most preferably of 75%, 85%, 90%, 95%, or greater. Reduce or inhibit can refer to the symptoms of the disorder being treated, e.g., the presence or amount of inflammation or ulcers.

An “effective amount” or “therapeutically effective amount” of an agent, e.g., a pharmaceutical formulation, refers to an amount effective, at dosages and for periods of time necessary, to achieve the desired therapeutic or prophylactic result.

As used herein, “treatment” (and grammatical variations thereof such as “treat” or “treating”) refers to clinical intervention in an attempt to alter the natural course of a disease in the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some aspects, antibodies of this disclosure are used to delay development of a disease or to slow the progression of a disease.

For example, with regard to IBD, “treatment” can refer to a decrease in the likelihood of developing IBD, a decrease in the rate of developing IBD, and a decrease in the severity of the disease. Those in need of treatment include those already with IBD as well as those in which IBD is to be prevented. Desirable effects of treatment include, but are not limited to, preventing occurrence or recurrence of disease, alleviating symptoms (e.g., diarrhea, fever, fatigue, abdominal pain, cramping, hematochezia, reduced appetite, and unintended weight loss), diminishing any direct or indirect pathological consequences of the disease, preventing the disease, decreasing the rate of disease progression, ameliorating or palliating the disease state, and causing remission or improved prognosis. In some embodiments, vixarelimab or a variant thereof is used to delay development of a disease or to slow the progression of a disease.

The term “active moderate to severe ulcerative colitis”, unless otherwise indicated, means having an Adapted Mayo score of 5 to 9, with an endoscopy subscore of at least 2.6. With regard to active moderate to severe ulcerative colitis, “treatment” can refer to one or more of (1) a decrease in the Modified Mayo Score (mMS); (2) a decrease in stool frequency; (3) a decrease in rectal bleeding; and (4) endoscopic improvement. In some embodiments, the decrease in mMS is a decrease from baseline in the Modified Mayo Score (mMS) of >=2. In some embodiments, the decrease in mMS is a >=30% reduction from baseline. In some embodiments, the decrease in mMS is a decrease from baseline in the Modified Mayo Score (mMS) of >=2 and >=30% reduction from baseline. In some embodiments, the decrease in rectal bleeding is a decrease in rectal bleeding subscore of >=1 or absolute rectal bleeding subscore of <=1. In some embodiments, the decrease in rectal bleeding is a decrease in rectal bleeding subscore of >=1. In some embodiments, the decrease in rectal bleeding results in an absolute rectal bleeding subscore of <=1. In some embodiments, the endoscopic improvement is measured at week 12. In some embodiments, the endoscopic improvement results in a Mayo endoscopy subscore of <=1 (score of 1 modified to exclude friability). In some embodiments, the endoscopic improvement results in an endoscopic remission (e.g., a Mayo endoscopy subscore of 0).

The term “advanced UC therapy” refers to biologic therapies and targeted small molecules that are used to treat ulcerative colitis. Examples of advanced therapies include, but are not limited to, anti-TNF antibodies (e.g., infliximab, adalimumab, golimumab), anti-integrin antibodies (e.g., vedolizumab, natalizumab); antiIL12/23 antibodies (e.g., ustekinumab), anti IL23 antibodies (e.g., Risankizumab-rzaa, mirikizumab-mrkz), JAK inhibitors (e.g., tofacitinib, upadacitinib), SIP receptor modulators (e.g., etrasimod). “Inadequate response” to an approved advanced therapy means persistent signs and symptoms of active disease despite an induction regimen of the advanced therapy.

“Baseline” refers to screening values prior to starting treatment. In some embodiments, baseline means screening values prior to starting treatment in a clinical trial (e.g., values determined during screening for clinical trial entry). Depending on the context used herein, “baseline” refers to values in screening test values, including results/values for endoscopy with biopsy and a full Mayo Score assessment.

“Clinical remission,” as used herein, refers to a modified Mayo Score (mMS) of ≤2, a stool frequency subscore≤1, a rectal bleeding score=0, and/or an endoscope subscore≤1, wherein the endoscopy subscore of 1 is modified to exclude friability. “Sustained remission” means clinical remission at both Week 12 and Week 48 of treatment. “Clinical response” means a decrease from baseline in the Modified Mayo Score (mMS) of >2 and ≥30% reduction from baseline; a decrease in stool frequency, a decrease in rectal bleeding subscore of ≥1 or absolute rectal bleeding subscore of ≤1, an endoscopic improvement at week 12; and/or an endoscopic remission.

“Endoscopic improvement,” as used herein, refers to a Mayo endoscopy subscore of ≤1 (score of 1 modified to exclude friability). “Endoscopic remission,” as used herein, refers to a Mayo endoscopy subscore of 0. While the Mayo endoscopy analysis includes friability, it was subsequently determined to be too variable and, thus, excluded from the endoscopic description of severity in subsequent analyses, such as the UCEIS (see, e.g., Travis SPL et al., Gut, 2012, vol. 61 (4): 535-542).

The terms “Mayo Clinic Score,” “MCS,” and “Mayo Score” are used interchangeably herein to refer to a scoring system for assessment of IBD (e.g., UC (e.g., active moderate to severe UC) or Crohn's disease), for example, as described in Schroeder et al. N. Engl. J. Med. 317 (26): 1625-1629, 1987, which is incorporated herein by reference in its entirety. The MCS includes four components: stool frequency, rectal bleeding, endoscopy findings, and a physician's global assessment. The stool frequency subscore is determined according to the following criteria: 0=normal number of stools for the subject; 1=1-2 more stools than normal; 2=3-4 stools more than normal; and 3=5 or more stools more than normal. The rectal bleeding subscore is determined according to the following criteria: 0=no blood seen; 1=streaks of blood with stool less than half the time; 2=obvious blood with stool most of the time; and 3=blood alone is passed. The endoscopy findings (“endoscopic”) subscore is determined according to the following criteria: 0=normal or inactive disease; 1=mild disease (erythema, decreased vascular pattern, mild friability); 2=moderate disease (marked erythema, absent vascular pattern, friability, erosions); and 3=severe disease (spontaneous bleeding, ulceration). The physician's global assessment is determined according to the following criteria: 0=normal; 1=mild disease; 2=moderate disease; and 3=severe disease. In some embodiments, the subject's MCS before treatment is about 3, about 4, about 5, about 6, about 7, about 8, about 9, about 10, about 11, or about 12. In some embodiments, the subject's MCS before treatment is 3, 4, 5, 6, 7, 8, 9, 10, 11, or 12.

The terms “modified MCS,” “mMCS,” “Modified Mayo Clinic Score,” “Modified Mayo Score,” and “mMS” are used interchangeably herein and refer to a composite of three MS assessments: stool frequency, rectal bleeding, and endoscopy findings. In other words, the mMS includes the stool frequency, rectal bleeding, and endoscopic subscores but omits the physician's global assessment subscore of the MS. In some embodiments, the subject's mMS before treatment is about 3, about 4, about 5, about 6, about 7, about 8, or about 9. In some embodiments, the subject's mMS before treatment is 3, 4, 5, 6, 7, 8, or 9.

The terms “Inflammatory Bowel Disease Questionnaire” and “IBDQ” refer to a scoring system for assessment of IBD (e.g., UC (e.g., moderate to severe UC) or Crohn's disease) as described, for example, in Irvine et al J. Pediatr. Gastroenterol. Nutr. 28 (4): S23-S27, 1999, which is incorporated herein by reference in its entirety. The IBDQ includes thirty-two questions scored on a 7-point scale from 1 (worst) to 7 (best) for a range of possible scores from 32 to 224.

The term “Robarts Histological Index” refers to a scoring system for assessment of IBD (e.g., UC (e.g., moderate to severe UC) or Crohn's disease) as described, for example, in Mosli et al. Gut 66 (1): 50-58, 2017, which is incorporated herein by reference in its entirety.

The terms “Ulcerative Colitis Endoscopic Index of Severity” and “UCEIS” refer to a scoring system for assessment of IBD (e.g., UC (e.g., moderate to severe UC) or Crohn's disease) as described, for example, in Travis et al. Gut 61:535-542, 2012, which is incorporated herein by reference in its entirety. The descriptors and definitions of the UCEIS are shown below in Table 1.

TABLE 1
UCEIS Descriptors and Definitions

| Descriptor (score | | |
|---|---|---|
| most severe | Likert scale | |
| lesions) | anchor points | Definition |
| Vascular pattern | Normal (1) | Normal vascular pattern with arborization of |
| | | capillaries clearly defined, or with blurring or patchy |
| | | loss of capillary margins |
| | Patchy | Patchy obliteration of vascular pattern |
| | obliteration (2) | |
| | Obliterated (3) | Complete obliteration of vascular pattern |
| Bleeding | None (1) | No visible blood |
| | Mucosal (2) | Some spots or streaks of coagulated blood on the |
| | | surface of the mucosa ahead of the scope, which can |
| | | be washed away |
| | Luminal mild (3) | Some free liquid blood in the lumen |
| | Luminal | Frank blood in the lumen ahead of endoscope or |
| | moderate or | visible oozing from mucosa after washing |
| | severe (4) | intraluminal blood, or visible oozing from a |
| | | hemorrhagic mucosa |
| Erosions and | None (1) | Normal mucosa, no visible erosions or ulcers |
| ulcers | Erosions (2) | Tiny (≤ 5mm) defects in the mucosa, of a white or |
| | | yellow color with a flat edge |
| | Superficial ulcer | Larger (>5 mm) defects in the mucosa, which are |
| | (3) | discrete fibrin-covered ulcers in comparison with |
| | | erosions, but remain superficial |
| | Deep ulcer (4) | Deeper excavated defects in the mucosa, with a |
| | | slightly raised edge |

The term “Nancy Histological Index” refers to a scoring system for assessment of IBD (e.g., UC (e.g., moderate to severe UC) or Crohn's disease) as described, for example, in Marchal-Bressenot et al. Gut 66 (1): 43-49, 2017, which is incorporated herein by reference in its entirety.

The “pathology” of a disease or condition includes all phenomena that compromise the well-being of the subject.

“Amelioration,” “ameliorating,” “alleviation,” “alleviating,” or equivalents thereof, refers to both therapeutic treatment and prophylactic or preventative measures, wherein the object is to ameliorate, prevent, slow down (lessen), decrease or inhibit a disease or condition, e.g., IBD (e.g., UC (e.g., moderate to severe UC) or Crohn's disease) or GVHD (e.g., acute or chronic GVHD, including intestinal GVHD). Those in need of treatment include those already with the disease or condition as well as those prone to having the disease or condition or those in whom the disease or condition is to be prevented.

“Administering” or “administration of” a substance, a compound or an agent to a subject refers to the contact of that substance, compound or agent to the subject or a cell, tissue, organ, or bodily fluid of the subject. For example, a compound or an agent can be administered intravenously or subcutaneously. Administering can also be performed, for example, once, a plurality of times, and/or over one or more extended periods. The administration may be either direct administration, including self-administration, or indirect administration, including the act of prescribing a drug. For example, as used herein, a physician who instructs a subject to self-administer a drug, or to have the drug administered by another and/or who provides a subject with a prescription for a drug is administering the drug to the subject. In some embodiments, a “combination” or a “combination therapy” refers to the administration of more than one therapeutic agent. When more than one substance, compound or agent is being administered, the administration can be simultaneous or sequential. “Simultaneous administration” refers to the administration of multiple therapeutic agents at the same time. The simultaneously administered therapeutic agents can be co-formulated or mixed prior to administration. “Sequential administration” refers to the administration of multiple therapeutic agents at different times in a manner that achieves overlapping results. For example, two therapeutic agents can be administered on same day in two separate injections. As an alternate example, one of the agents can be injected on one day, and the second can be injected on a subsequent day.

As used herein, the term “diagnosis” refers to the identification or classification of a molecular or pathological state, disease or condition. For example, “diagnosis” can refer to identification of a particular type of a condition (such ulcerative colitis or Crohn's disease). “Diagnosis” can also refer to the classification of a particular subtype of a condition, e.g., by histopathological or radiographic criteria or by molecular features (e.g., a subtype characterized by expression of one or a combination of particular genes or proteins encoded by the genes). The term “suffering from” or “experiencing” as used herein can refer to a subject who has not received a formal diagnosis of a disease or disorder but displays several of the symptoms which may lead to a formal diagnosis of the disease or disorder.

As used herein, the term “aiding diagnosis” refers to methods that assist in making a clinical determination regarding the presence, or nature, of a particular type of symptom or condition of a condition (such as ulcerative colitis). For example, a method of aiding diagnosis of a condition (such as ulcerative colitis) can include measuring the expression of certain genes in a biological sample from an individual.

As used herein, the term “prognosis” is used herein to refer to the prediction of the likelihood of survival over time as well as one or more disease symptoms attributable to a condition (such as ulcerative colitis) worsening over time.

“Loss of response to an advanced therapy” means recurrence of symptoms during maintenance dosing of advanced therapy following prior clinical benefit. Discontinuation despite clinical benefit or discontinuation due to loss of access does not qualify as loss of response.

“Intolerance to an advanced therapy” means history of adverse effect necessitating treatment discontinuation of the advanced therapy (including, but not limited to infusion-related reaction, demyelination, congestive heart failure, and infection). A subject with “advanced failure” is defined as a subject having inadequate response, loss of response to, or intolerance to up to two prior classes of approved advanced therapies.

The term “conventional ulcerative colitis therapy” refers to immunosuppressant and/or corticosteroid treatment. Conventional therapies for ulcerative colitis and Crohn's disease (CD) include, but are not limited to, aminosalicylates, corticosteroids, thiopurines, and methotrexate. Examples of aminosalicylates include, but are not limited to, 5-aminosalicylic acid, sulfasalazine, balsalazide, olsalazine and mesalazine, in forms like Eudragit-S-coated, pH-dependent mesalamine, ethylcellulose-coated mesalamine, and multimatrix-release mesalamine. A subject with “conventional failure” is defined as a subject having inadequate response, loss of response to, or intolerance to prior conventional UC therapies. Failure of only 5-ASA treatment is not sufficient.

“Inadequate response, loss of response, or intolerance to corticosteroid treatment” is defined as one or more of: steroid refractory (defined as persistent symptoms of active disease despite treatment with at least one 4-week induction regimen that included greater than or equal to 30 mg/day of oral prednisone (or equivalent) for at least 2 weeks or greater than or equal to 30 mg/day prednisone (or equivalent) for at least one week; steroid dependent (defined as two failed attempts to taper steroids below a dose equivalent to 10 mg/day or oral prednisone (or equivalent); and steroid intolerant (defined as history of intolerance to corticosteroids (including, but not limited to, Cushing's syndrome, osteopenia/osteoporosis, hyperglycemia, insomnia or infection). Examples of corticosteroids include, but are not limited to, prednisone.

“Inadequate response to, loss of response to, or intolerance to prior immunosuppressant treatment” is defined as persistent signs and symptoms of active disease despite treatment with at least one 12-week regimen of azathioprine (AZA) (greater than or equal to 1.5 mg/day) or 6-MP (greater than or equal to 0.75 mg/day) and/or methotrexate (MTX) (greater than or equal to 15 mg/week); persistent signs and symptoms of active disease despite a 6-thioguanine nucleotide level of greater than or equal to 230 pmol/8×10⁸ red blood cells (RBCs) during at least one 12-week regimen of oral AZA or 6-MP at a stable or increasing dose; and history of intolerance to AZA, 6-MP or MTX (including, but not limited to, nausea/vomiting, abdominal pain, pancreatitis, liver function test abnormalities, lymphopenia, TPMT genetic mutation, or infection).

As used herein, “induction therapy” is an initial stage of therapy, wherein a subject is administered a relatively intensive dosing regimen of a therapeutic agent. The therapeutic agent, e.g., antibody, is administered in a way that quickly provides an effective amount of the agent suitable for certain purposes, such as, e.g., for inducing a clinical response and ameliorating disease symptoms. In some embodiments herein, the induction therapy results in active moderate-to-severe UC clinical remission, active moderate-to-severe UC clinical response, and/or method results in improved signs or improved symptoms. In some embodiments, the improved signs or improved symptoms is an improvement in ulcerative colitis bowel movement signs and symptoms defined as the proportion of subjects with a greater than or equal to 6-point decrease in the UC-PRO/SS bowel domain score. In some embodiments, the improved signs or improved symptoms is a change from baseline in UC functional signs and symptoms as assessed by IC-PRO/SS score. In some embodiments, the improved signs or improved symptoms is an improvement in ulcerative colitis functional symptoms as defined by proportion of subjects with a greater than or equal to 2-point increase in the UC-PRO/SS functional domain score. In some embodiments, the improved signs or improved symptoms is assessed at week 12 and week 48. In some embodiments, the treatment with a therapeutically effective dose of vixarelimab for at least 48 weeks results in a sustained remission.

As used herein, the term “maintenance” dose herein refers to one or more doses of a therapeutic agent administered to the subject over a treatment period. Usually, the maintenance doses are administered at spaced treatment intervals, such as approximately every week, approximately every 2 weeks, approximately every 3 weeks, or approximately every 4 weeks, preferably every 3 weeks. An exemplary maintenance dose for subcutaneous vixarelimab is 720 mg. An exemplary dosing frequency is every 2 weeks or every 4 weeks.

A “patient population” refers to a group of IBD (e.g., UC) patients. Such populations can be used to demonstrate statistically significant efficacy and/or safety of a drug, such as vixarelimab.

“Safety data” refers to the data obtained in a controlled clinical trial showing the prevalence and severity of adverse events to guide the user regarding the safety of the drug, including guidance on how to monitor and prevent adverse reactions to the drug. “Efficacy data” refers to the data obtained in controlled clinical trial showing that a drug effectively treats a disease, such as IBD (e.g., UC).

As used herein, the term “sample” refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. A “tissue” or “cell sample” refers to a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample can also be primary or cultured cells or cell lines. Optionally, the tissue or cell sample is obtained from a disease tissue/organ. The tissue sample can contain compounds which are not naturally intermixed with the tissue in nature such as preservatives, anticoagulants, buffers, fixatives, nutrients, antibiotics, and the like.

As used herein, the terms “control,” “control cohort,” “reference sample,” “reference cell,” “reference tissue,” “control sample,” “control cell,” and “control tissue” refer to a sample, cell or tissue obtained from a source that is known, or believed, to not be afflicted with the disease or condition for which a method or composition of the present disclosure is being used to identify. The control can include one control or multiple controls. In one embodiment, a reference sample, reference cell, reference tissue, control sample, control cell, or control tissue is obtained from a healthy part of the body of the same subject or patient in whom a disease or condition is being identified using a composition or method of the present disclosure. In one embodiment, a reference sample, reference cell, reference tissue, control sample, control cell, or control tissue is obtained from a healthy part of the body of an individual who is not the subject or patient in whom a disease or condition is being identified using a composition or method of the present disclosure.

The term “antibody” is used herein in the broadest sense and encompasses various antibody structures, including but not limited to monoclonal antibodies, polyclonal antibodies, multi-specific antibodies (e.g., bispecific antibodies), and antibody fragments so long as they exhibit the desired antigen-binding activity. Such antibodies can be chimeric, humanized, human and synthetic.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6th ed., W. H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al., supra. In one aspect, for the VH, the subgroup is subgroup III as in Kabat et al., supra.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. Antibodies produced by host cells, however, may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case in particular where the final two C-terminal amino acids of the heavy chain are glycine (G446) and lysine (K447, Kabat EU numbering). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal lysine if not indicated otherwise. The corresponding sequence including a C-terminal lysine residue is also encompassed, however. Accordingly, in one aspect, a heavy chain including an Fc region as specified herein comprises an additional C-terminal lysine residue (K447, Kabat EU numbering). Also encompassed is the corresponding sequence without the C-terminal glycine residue. Accordingly, in one aspect, a heavy chain including an Fc region as specified herein lacks the C-terminal glycine residue (G446, Kabat EU numbering). In such a heavy chain, the C-terminal amino acid residue may be proline (P445, Kabat EU numbering) or proline amide (P445—NH₂, Kabat EU numbering). Unless otherwise specified herein, numbering of amino acid residues in the Fc region or heavy chain constant region is according to the EU numbering system, also called the EU index, as described in Kabat 1991.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, MegAlign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein: protein) program and default options (BLOSUM50; open: −10; ext: −2; Ktup=2) to ensure a global, rather than local, alignment is performed. Percent amino acid identity is given in the output alignment header.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

As used herein, the terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids. Trans-splicing, polypeptide cleavage and polypeptide ligation can also be involved in expression of a protein in a cell. Methods for polynucleotide and polypeptide delivery to cells are known in the art.

The term “package insert” is used to refer to instructions customarily included in commercial packages of therapeutic products, that contain information about the indications, usage, dosage, administration, combination therapy, contraindications and/or warnings concerning the use of such therapeutic products.

### DETAILED DESCRIPTION

There is an ongoing high unmet medical need in the treatment of inflammatory bowel disease, including in patients with UC. Despite advances in the range of therapeutic options from biologics to targeted small molecules, many patients have an inadequate response to therapy, lose response over time, or cannot tolerate available treatments. In addition to inadequate disease control, available treatments are associated with at least one or more risks such as serious infections and thromboembolic, cardiovascular, and malignancy. Because of the limitations of currently available therapies, there exists a need for new safe and effective UC treatment options, especially in patients for whom prior therapies have failed. Vixarelimab is being developed as a novel therapeutic agent to achieve clinical remission in patients diagnosed with inflammatory bowel disease such as patients with CD or with moderate to severe UC who have demonstrated inadequate response to, loss of response to, or intolerance to prior conventional or advanced therapy.

### Vixarelimab and Inhibition of the OSM/OSMRβ Pathway

### Vixarelimab

Vixarelimab is a fully human, monoclonal antibody that targets OSMRβ (oncostatin M (OSM) receptor) (described as “Ab2” in U.S. Pat. No. 9,593,163 (herein, the “163 patent”), the contents of which are incorporated herein by reference in their entirety). OSMRβ is a cytokine receptor subunit that heterodimerizes with IL-31 receptor alpha (IL-31Ra) or gp130 to form two distinct receptors for two distinct cytokines, interleukin-31 (IL-31) and OSM, respectively, each of them mediating signaling pathways implicated in inflammation and fibrosis (Mozaffarian et al. 2008; Marden et al. 2020; Yaseen et al. 2020; Kuzumi et al. 2021). The heavy and light chain sequences of vixarelimab are provided in Table 2 below. Vixarelimab is also known in some non-patent publications as KPL-716. In preferred embodiments, the preferred anti-OSMRβ antibody for treatment of inflammatory bowel disease (e.g., ulcerative colitis or Crohn's disease) as described herein is vixarelimab.

TABLE 2
Amino Acid sequences

| | SEQ | |
|---|---|---|
| | ID | |
| Vixarelimab | NO | Sequence |
| Vixarelimab | 1 | QVQLVQSGAEVKKPGASVKVSCKASGYTFTSYEINWVRQA |
| Heavy chain | | TGQGLEWMGWMNPNSGYTGYAQKFQGRVTMTRDTSISTA |
| | | YMEMSSLRSEDTAVYYCARDIVAANTDYYFYYGMDVWGQ |
| | | GTTVTVSSASTKGPSVFPLAPCSRSTSESTAALGCLVKDYFP |
| | | EPVTVSWNSGALTSGVHTFPAVLQSSGLYSLSSVVTVPSSSL |
| | | GTKTYTCNVDHKPSNTKVDKRVESKYGPPCPPCPAPEFLGG |
| | | PSVFLFPPKPKDTLMISRTPEVTCVVVDVSQEDPEVQFNWY |
| | | VDGVEVHNAKTKPREEQFQSTYRVVSVLTVLHQDWLNGK |
| | | EYKCKVSNKGLPSSIEKTISKAKGQPREPQVYTLPPSRDELT |
| | | KNQVSLTCLVKGFYPSDIAVEWESNGQPENNYKTTPPVLDS |
| | | DGSFFLYSKLTVDKSRWQQGNVFSCSVMHEALHNHYTQKS |
| | | LSLSPG |
| Vixarelimab | 2 | QSVLTQPPSASGTPGQRVTISCSGSNSNIGSNTVNWYHQLPG |
| Light chain | | TAPKLLIYNINKRPSGVPDRFSGSKSGSSASLAISGLQSEDEA |
| | | DYYCSTWDDSLDGVVFGGGTKLTVLGQPKAAPSVTLFPPSS |
| | | EELQANKATLVCLISDFYPGAVTVAWKADSSPVKAGVETTT |
| | | PSKQSNNKYAASSYLSLTPEQWKSHRSYSCQVTHEGSTVEK |
| | | TVAPTECS |
| Vixarelimab | 3 | QVQLVQSGAEVKKPGASVKVSCKASGYTFTSYEINWVRQA |
| heavy chain | | TGQGLEWMGWMNPNSGYTGYAQKFQGRVTMTRDTSISTA |
| variable domain | | YMEMSSLRSEDTAVYYCARDIVAANTDYYFYYGMDVWGQ |
| | | GTTVTVSS |
| Vixarelimab | 4 | QSVLTQPPSASGTPGQRVTISCSGSNSNIGSNTVNWYHQLPG |
| Light chain | | TAPKLLIYNINKRPSGVPDRFSGSKSGSSASLAISGLQSEDEA |
| variable domain | | DYYCSTWDDSLDGVVFGGGTKLTVLG |

### Vixarelimab for Treatment of Inflammatory Bowel Disease (IBD)

Despite the emergence of new agents to treat patients with UC, additional treatment options are needed to improve disease control and outcomes, particularly targeting novel mechanisms in the pathophysiology of UC. While prior clinical studies of vixarelimab in patients suffering from prurigo nodularis (PN) have shown safety and some efficacy, there are no clinical data describing treatment of IBD (e.g., ulcerative colitis) patients with an anti-OSMRβ antibody, such as vixarelimab, which blocks both OSM and IL-31 signaling. Importantly, dosing of the antibody to achieve therapeutic activity in patients suffering from inflammatory bowel disease remains a challenge considering the unknown impact of anti-OSMRβ antibody exposure to OSMRβ on the surface of cells in the intestinal vs. skin environment. Indeed, translation from IL-31-driven pruritic indication to OSM-driven inflammation in the intestine is unpredictable.

Oncostatin M signaling is initiated by binding to type I or type II receptor complexes and involves activation of JAK/STAT, MAPK and PI3K. The type I receptor comprises gp 130 and Leukemia Inhibitory Factor receptor (LIFR), the type II receptor comprises gp130 and Oncostatin M Receptor (OSMR). OSM initially binds with low affinity to gp130 and then recruits either LIFR (type I) or OSMR (type II). LIFR, OSMR, and gp130 all associate with members of the Jak family of tyrosine kinases through their cytoplasmic domains, leading to the phosphorylation, dimerization, and nuclear translocation of STAT proteins, predominantly STAT3, STAT5, and STAT1. Without being bound by theory, it is considered that blocking OSM activation of the type II receptor without inhibiting the type I OSMRβ receptor may provide a better safety profile in patients receiving the anti-OSMRβ antibody as described herein. For example, binding of the therapeutic antibody to the OSMRβ subunit of the type II receptor allows continued signaling of OSM through the type I receptor. In some embodiments, administration of an anti-OSMRβ antibody to a subject suffering from an inflammatory bowel disease (e.g., ulcerative colitis) does not elicit more than a mild case of anemia or does not cause unsafe elevation of thrombopoietin and/or erythropoietin.

Accordingly, the present disclosure provides methods for administering vixarelimab, or another anti-OSMRβ antibody which inhibits both OSM and IL-31 signaling, to treat an inflammatory bowel disease, including Crohn's Disease (CD) or ulcerative colitis (CD).

A phase II, multicenter induction study with an active treatment extension (ATE) is described to demonstrate efficacy and safety of vixarelimab in the treatment of patients with moderate to severe ulcerative colitis. It is understood that demonstrated efficacy and safety can be applicable for use in treating patients mild, moderate, or severe UC as well as in patients with other inflammatory bowel diseases, such as Crohn's disease.

In some embodiments, the patients have demonstrated inadequate response (IR) to, loss of response to, or intolerance to prior advanced therapy for an inflammatory disease. In some embodiments, the subject has inadequate response to, loss of response to, or intolerance to up to two prior classes of approved ulcerative colitis advanced therapies and/or inadequate response to, loss of response to, or intolerance to prior conventional ulcerative colitis therapies and/or inadequate response to, loss of response to, or intolerance to prior immunosuppressant treatment. In some embodiments, the subject has inadequate response to, loss of response to, or intolerance to up to two prior classes of approved ulcerative colitis advanced therapies. In some embodiments, the subject has inadequate response to, loss of response to, or intolerance to prior conventional ulcerative colitis therapies. In some embodiments, the subject has inadequate response to, loss of response to, or intolerance to prior immunosuppressant treatment. In some embodiments, combination therapy with vixarelimab and a therapeutic anti-TNF antibody will be efficacious without an increase in adverse events as compared to treatment with the anti-TNF antibody alone.

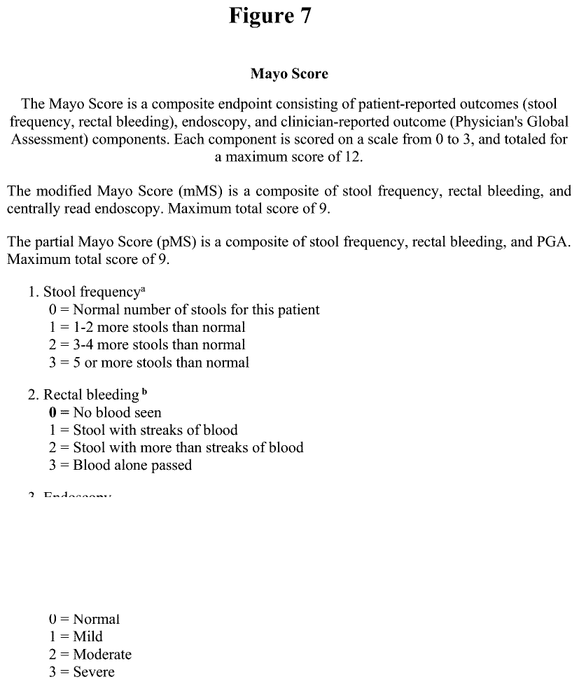

Therapeutic efficacy of treatment of ulcerative colitis by administration of vixarelimab can be assessed, for example, using the Mayo Score, the Modified Mayo Score (mMS), and the Ulcerative Colitis Disease Activity Index (UCDAI), each well known to the clinical practitioner in the field of IBD (see FIG. 7).

UC is a form of colitis, an inflammatory disease of the intestine, usually the colon, which includes characteristic ulcers. Symptoms of active disease usually include diarrhea mixed with blood, usually accompanied with varying degrees of abdominal pain, from mild discomfort to severely painful cramps. There are a number of methods for assessing the severity of disease, including the Mayo Score, the Modified Mayo Score (mMS) and Ulcerative Colitis Disease Activity Index (UCDAI).

Efficacy of treatment with vixarelimab can be assessed using the modified Mayo Score (mMS), with the Mayo endoscopic subscore calculated on the basis of centrally read endoscopy. The mMS and partial Mayo Score (pMS) can be derived from the Mayo Score.

The Mayo Score is a composite endpoint consisting of patient-reported outcomes (stool frequency, rectal bleeding), endoscopy, and clinician-reported outcome (Physician's Global Assessment) components. Each component is scored on a scale from 0 to 3 and totaled for a maximum score of 12.

The modified Mayo Score (mMS) is a composite of stool frequency, rectal bleeding, and centrally read endoscopy. Maximum total score of 9.

The partial Mayo Score (pMS) is a composite of stool frequency, rectal bleeding, and PGA. Maximum total score of 9.

Patients deemed eligible for the phase 2 study descripted herein can be those with active moderate to severe UC with an mMS of 5-9 and endoscopic score of at least 2, consistent with regulatory guidance (U.S. Food and Drug Administration [FDA] 2022). These patients may benefit from the anticipated therapeutic effects of vixarelimab. All patients considered for participation will have a diagnosis of moderate to severe UC established at least 3 months prior to screening, with active disease confirmed by clinical and endoscopic evidence during screening. In some embodiments, patients have demonstrated an inadequate response, loss of response, or intolerance to prior conventional UC therapies and/or to up to 2 prior classes of approved advanced therapies (as defined in Example 2 below).

As described herein, PK/PD modeling was used to predict a therapeutically effective dose of an anti-OSMRβ antibody for treatment of intestinal disorders such as UC or CD. The modeling relied in part on in vitro potency assays, preclinical PK/PD studies, and favorable PK and safety data from doses tested in phase 1 and 2 trials with AD and PN patients (see, e.g., Example 1 below). The present disclosure provides methods for treating an inflammatory bowel disease (e.g., ulcerative colitis) by administering to a subject in need thereof an anti-OSMRβ wherein the anti-OSMRβ antibody binds the extracellular domain of the OSMRβ protein and blocks signaling of the type II OSMR by both OSM and IL31. In preferred embodiments, the anti-OSMRβ antibody is vixarelimab and the dosing regimen for administering vixarelimab is 720 mg every 2 weeks or every 4 weeks.

### Anti-OSMRβ Antibodies for The Treatment of Active Moderate to Severe Ulcerative Colitis

In another aspect, this disclosure provides method for treating ulcerative colitis comprising administering to a subject in need thereof a therapeutically effective dose of an anti-oncostatin M receptor (OSMR) β antibody. In some embodiments, the anti-OSMRβ antibody comprises a heavy chain variable domain (VH) comprising SEQ ID NO: 3 and a light chain variable domain (VL) comprising SEQ ID NO: 4. In some embodiments, the anti-OSMRβ antibody comprises a heavy chain variable domain (VH) comprising SEQ ID NO: 3. In some embodiments of this disclosure, the anti-OSMRβ antibody comprises a heavy chain variable domain (VL) comprising SEQ ID NO: 4. In some embodiments, the anti-OSMRβ antibody is vixarelimab.

In another aspect, the disclosure provides an anti-oncostatin M receptor (OSMR) B antibody for use in treating ulcerative colitis comprising administering to a subject in need, wherein the anti-OSMRβ antibody comprises a heavy chain variable domain (VH) comprising SEQ ID NO: 3 and a light chain variable domain (VL) comprising SEQ ID NO: 4. In some embodiments, the anti-OSMRβ antibody is vixarelimab.

In a further aspect, the disclosure provides use of an anti-OSMRβ antibody in the manufacture of a pharmaceutical composition for treating ulcerative colitis comprising administering to a subject in need, wherein the anti-OSMRβ antibody comprises a heavy chain variable domain (VH) comprising SEQ ID NO: 3 and a light chain variable domain (VL) comprising SEQ ID NO: 4. In some embodiments, the anti-OSMRβ antibody is vixarelimab.

In some embodiments of any of the above aspects, the therapeutically effective dose is about 720 mg of the anti-OSMRβ antibody (e.g., vixarelimab). In some embodiments of any of the above aspects, the therapeutically effective dose is 720 mg of the anti-OSMRβ antibody (e.g., vixarelimab). In some embodiments of any of the above aspects, the anti-OSMRβ antibody (e.g., vixarelimab) is administered for at least 12 weeks. In some embodiments of any of the above aspects, the therapeutically effective dose is about 720 mg of the anti-OSMRβ antibody (e.g., vixarelimab) for at least 12 weeks. In some embodiments of any of the above aspects, the therapeutically effective dose is 720 mg of the anti-OSMRβ antibody (e.g., vixarelimab) for at least 12 weeks. In some embodiments of any of the above aspects, the anti-OSMRβ antibody (e.g., vixarelimab) is administered at weeks 0, 1, 2, and every 2 weeks (Q2W) thereafter. In some embodiments of any of the above aspects, the therapeutically effective dose is administered at weeks 0, 1, 4, and every 4 weeks (Q4W) thereafter. In some embodiments of any of the above aspects, the anti-OSMRβ antibody (e.g., vixarelimab) is administered at week 0. In some embodiments, the anti-OSMRβ antibody (e.g., vixarelimab) is administered at week 1. In some embodiments of any of the above aspects, the anti-OSMRβ antibody (e.g., vixarelimab) is administered at week 2. In some embodiments of any of the above aspects, the anti-OSMRβ antibody (e.g., vixarelimab) is administered at week 4. In some embodiments of any of the above aspects, the anti-OSMRβ antibody (e.g., vixarelimab) is administered every 2 weeks (Q2W). In some embodiments of any of the above aspects, the anti-OSMRβ antibody (e.g., vixarelimab) is administered every 4 weeks (Q4W). In some embodiments of any of the above aspects, the anti-OSMRβ antibody (e.g., vixarelimab) is administered for 48 weeks. In some embodiments of any of the above aspects, the anti-OSMRβ antibody (e.g., vixarelimab) is administered intravenously. In some embodiments of any of the above aspects, the anti-OSMRβ antibody (e.g., vixarelimab) is administered subcutaneously.

In some embodiments of any of the above aspects, the therapeutically effective dose of about 720 mg of the anti-OSMRβ antibody (e.g., vixarelimab) is subcutaneously administered. In some embodiments of any of the above aspects, the therapeutically effective dose of 720 mg of the anti-OSMRβ antibody (e.g., vixarelimab) is subcutaneously administered. In some embodiments of any of the above aspects, the anti-OSMRβ antibody (e.g., vixarelimab) is subcutaneously administered for at least 12 weeks. In some embodiments of any of the above aspects, the therapeutically effective dose of about 720 mg of the anti-OSMRβ antibody (e.g., vixarelimab) is subcutaneously administered for at least 12 weeks. In some embodiments of any of the above aspects, the therapeutically effective dose of 720 mg of the anti-OSMRβ antibody (e.g., vixarelimab) is subcutaneously administered for at least 12 weeks. In some embodiments of any of the above aspects, the anti-OSMRβ antibody (e.g., vixarelimab) is subcutaneously administered at weeks 0, 1, 2, and every 2 weeks (Q2W) thereafter. In some embodiments of any of the above aspects, the therapeutically effective dose is subcutaneously administered at weeks 0, 1, 4, and every 4 weeks (Q4W) thereafter. In some embodiments of any of the above aspects, the anti-OSMRβ antibody (e.g., vixarelimab) is subcutaneously administered at week 0. In some embodiments, the anti-OSMRβ antibody (e.g., vixarelimab) is subcutaneously administered at week 1. In some embodiments of any of the above aspects, the anti-OSMRβ antibody (e.g., vixarelimab) is subcutaneously administered at week 2. In some embodiments of any of the above aspects, the anti-OSMRβ antibody (e.g., vixarelimab) is subcutaneously administered at week 4. In some embodiments of any of the above aspects, the anti-OSMRβ antibody (e.g., vixarelimab) is subcutaneously administered every 2 weeks (Q2W). In some embodiments of any of the above aspects, the anti-OSMRβ antibody (e.g., vixarelimab) is subcutaneously administered every 4 weeks (Q4W). In some embodiments of any of the above aspects, the anti-OSMRβ antibody (e.g., vixarelimab) is administered for 48 weeks.

In some embodiments of any of the above aspects, the anti-OSMRβ comprises a heavy chain (HC) comprising SEQ ID NO: 1. In some embodiments of any of the above aspects, the anti-OSMRβ comprises a light chain (LC) comprising SEQ ID NO: 2. In some embodiments of any of the above aspects, the anti-OSMRβ comprises a heavy chain (HC) comprising SEQ ID NO: 1 and a light chain (LC) comprising SEQ ID NO: 2. In some embodiments of any of the above aspects, the anti-OSMRβ is vixarelimab.

In some embodiments of any of the above aspects, the administering with a therapeutically effective dose of the anti-OSMRβ antibody (e.g., vixarelimab) for at least 12 weeks results in clinical remission in the patient.

In some embodiments, the clinical remission in the subject is demonstrated by a modified Mayo Score (mMS) of ≤2. In some embodiments, the clinical remission in the subject is demonstrated by a stool frequency subscore≤1. In some embodiments, the clinical remission in the subject is demonstrated by a rectal bleeding score=0. In some embodiments, the clinical remission in the subject is demonstrated by an endoscope subscore≤1, wherein the endoscopy subscore of 1 is modified to exclude friability. In other embodiments, the clinical remission in the subject is demonstrated by one or more of mMS of ≤2, a stool frequency subscore≤1, a rectal bleeding score=0, and an endoscope subscore≤1, wherein the endoscopy subscore of 1 is modified to exclude friability. In other embodiments, the clinical remission in the subject is demonstrated by two or more of mMS of ≤2, a stool frequency subscore≤1, a rectal bleeding score=0, and an endoscope subscore≤1, wherein the endoscopy subscore of 1 is modified to exclude friability. In other embodiments, the clinical remission in the subject is demonstrated by three or more of mMS of ≤2, a stool frequency subscore≤1, a rectal bleeding score=0, and an endoscope subscore≤1, wherein the endoscopy subscore of 1 is modified to exclude friability. In still other embodiments, the clinical remission in the subject is demonstrated by mMS of ≤2, a stool frequency subscore≤1, a rectal bleeding score=0, and an endoscope subscore≤1, wherein the endoscopy subscore of 1 is modified to exclude friability.

In some embodiments of any of the above aspects, the administering with a therapeutically effective dose of the anti-OSMRβ antibody (e.g., vixarelimab) for at least 12 weeks results in a clinical response in the patient.

In some embodiments, the clinical response in the subject is demonstrated by the subject experiencing a decrease from baseline in the mMS of ≥2 and ≥30% reduction from baseline. In some embodiments, the clinical response in the subject is demonstrated by a reduction in stool frequency. In some embodiments, the clinical response in the subject is demonstrated by a decrease in rectal bleeding subscore of ≥1 or absolute rectal bleeding subscore of ≤1. In some embodiments, the clinical response in the subject is demonstrated by an endoscopic improvement at week 12 wherein endoscopic improvement is defined as a Mayo endoscopy subscore of ≤1 (score of 1 modified to exclude friability). In some embodiments, the clinical response in the subject is demonstrated by an endoscopic remission wherein endoscopic remission is defined as a Mayo endoscopy subscore of 0. In some embodiments, the clinical response in the subject is demonstrated by one or more of the subject experiencing a decrease from baseline in the mMS of >2 and ≥30% reduction from baseline, a decrease in rectal bleeding subscore of ≥1 or absolute rectal bleeding subscore of ≤1, an endoscopic improvement at week 12 wherein endoscopic improvement is defined as a Mayo endoscopy subscore of ≤1 (score of 1 modified to exclude friability), and/or an endoscopic remission wherein endoscopic remission is defined as a Mayo endoscopy subscore of 0. In some embodiments, the clinical response in the subject is demonstrated by two or more of the subject experiencing a decrease from baseline in the mMS of ≥2 and ≥30% reduction from baseline, a decrease in rectal bleeding subscore of ≥1 or absolute rectal bleeding subscore of ≤1, an endoscopic improvement at week 12 wherein endoscopic improvement is defined as a Mayo endoscopy subscore of ≤1 (score of 1 modified to exclude friability), and/or an endoscopic remission wherein endoscopic remission is defined as a Mayo endoscopy subscore of 0. In some embodiments, the clinical response in the subject is demonstrated by three or more of the subject experiencing a decrease from baseline in the mMS of ≥2 and ≥30% reduction from baseline, a decrease in rectal bleeding subscore of ≥1 or absolute rectal bleeding subscore of ≤1, an endoscopic improvement at week 12 wherein endoscopic improvement is defined as a Mayo endoscopy subscore of ≤1 (score of 1 modified to exclude friability), and/or an endoscopic remission wherein endoscopic remission is defined as a Mayo endoscopy subscore of 0. In other embodiments, the clinical response in the subject is demonstrated by the subject experiencing a decrease from baseline in the mMS of ≥2 and ≥30% reduction from baseline, a decrease in rectal bleeding subscore of ≥1 or absolute rectal bleeding subscore of ≤1, an endoscopic improvement at week 12 wherein endoscopic improvement is defined as a Mayo endoscopy subscore of ≤1 (score of 1 modified to exclude friability), and an endoscopic remission wherein endoscopic remission is defined as a Mayo endoscopy subscore of 0.

In some embodiments, the clinical response in the subject is demonstrated by the subject experiencing a decrease from baseline in the mMS of ≥2 and ≥30% reduction from baseline, a decrease in rectal bleeding subscore of ≥1 and absolute rectal bleeding subscore of ≤1.

In some embodiments, the clinical response in the subject is further demonstrated by the subject experiencing an endoscopic improvement at week 12 wherein endoscopic improvement is defined as a Mayo endoscopy subscore of ≤1 (score of 1 modified to exclude friability), and/or an endoscopic remission wherein endoscopic remission is defined as a Mayo endoscopy subscore of 0.

In some embodiments of any of the above aspects, the subject has inadequate response to, loss of response to, or intolerance to up to two prior classes of approved ulcerative colitis advanced therapies and/or inadequate response to, loss of response to, or intolerance to prior conventional ulcerative colitis therapies and/or inadequate response to, loss of response to, or intolerance to prior immunosuppressant treatment. In some embodiments of any of the above aspects, the subject has inadequate response to, loss of response to, or intolerance to up to two prior classes of approved ulcerative colitis advanced therapies. In some embodiments of any of the above aspects, the subject has inadequate response to, loss of response to, or intolerance to prior conventional ulcerative colitis therapies. In some embodiments of any of the above aspects, the subject has inadequate response to, loss of response to, or intolerance to prior immunosuppressant treatment.

Inadequate response to, loss of response to, or intolerance to prior immunosuppressant treatment in the subject is demonstrated by persistent signs and symptoms of active disease despite treatment with at least one 12-week regimen of AZA (greater than or equal to 1.5 mg/day) or 6-MP (greater than or equal to 0.75 mg/day) and/or methotrexate (MTX) (greater than or equal to 15 mg/week); persistent signs and symptoms of active disease despite a 6-thioguanine nucleotide level of greater than or equal to 230 pmol/8×10⁸ red blood cells (RBCs) during at least one 12-week regimen of oral AZA or 6-MP at a stable or increasing dose; and history of intolerance to AZA, 6-MP or MTX (including, but not limited to, nausea/vomiting, abdominal pain, pancreatitis, liver function test abnormalities, lymphopenia, TPMT genetic mutation, or infection).

Inadequate response to, loss of response, or intolerance to corticosteroid treatment in the subject is demonstrated by one or more of: steroid refractory (defined as persistent symptoms of active disease despite treatment with at least one 4-week induction regimen that included greater than or equal to 30 mg/day of oral prednisone (or equivalent) for at least 2 weeks or greater than or equal to 30 mg/day prednisone (or equivalent) for at least one week; steroid dependent (defined as two failed attempts to taper steroids below a dose equivalent to 10 mg/day or oral prednisone (or equivalent); and steroid intolerant (defined as history of intolerance to corticosteroids (including, but not limited to, Cushing's syndrome, osteopenia/osteoporosis, hyperglycemia, insomnia or infection). Examples of corticosteroids include, but are not limited to, prednisone.

In some embodiments of any of the above aspects, the subject is receiving an anti-tumor necrosis factor (anti-TNF) therapy. The anti-TNF therapy may be an anti-TNFα antibody, such as infliximab, adalimumab, golimumab, or a biosimilar thereof.

In some embodiments of any of the above aspects, the anti-OSMRβ antibody is administered in combination with a second therapeutic agent. In other embodiments, the second therapeutic agent is a therapeutic agent indicated for an inflammatory bowel disease. In some embodiments, the second therapeutic agent is an anti-TNFα antibody. In some embodiments, the anti-TNFα antibody is infliximab, adalimumab, golimumab, or a biosimilar thereof. In some embodiments, the second therapeutic agent is an anti-inflammatory agent. In some embodiments, the anti-inflammatory is 5-aminosalicylic acid or a corticosteroid. In some embodiments, the second therapeutic agent is an immunosuppressant. In some embodiments, the immunosuppressant is azathioprine, methotrexate, or 6-mercaptopurine. In some embodiments, the second therapeutic agent is an anti-IL-6 antibody or an anti-IL-6 receptor antibody.

In some embodiments, the anti-OSMRβ antibody is administered before, during, or after administration with the second therapeutic agent.

In some embodiments, the anti-OSMRβ antibody is administered to the subject after the subject has been treated with the second therapeutic agent for at least 1 week, 1 month, 6 months, 1 year, 3 years, or 5 years.

In some embodiments of any of the above aspects, the subject has not been diagnosed with or is not experiencing a lung fibrotic disease or disorder. In some embodiments of any of the above aspects, the subject has not been diagnosed with or is not experiencing a fibrotic skin disease such as PN or AD.

### Pharmaceutical Compositions

In a further aspect, the disclosure provides pharmaceutical compositions comprising vixarelimab or a variant thereof is provided herein, e.g., for use in any of the therapeutic methods disclosed herein. In some embodiments, the pharmaceutical composition comprises comprising vixarelimab or a variant thereof and a pharmaceutically acceptable carrier. In some embodiments, the pharmaceutical composition comprises any of the antibodies provided herein and at least one additional therapeutic agent, e.g., as described below.

Pharmaceutical compositions (formulations) comprising vixarelimab or a variant thereof can be prepared by combining the antibody with pharmaceutically acceptable carriers or excipients known to the skilled person. See, for example, Remington's Pharmaceutical Sciences 16th edition, Osol, A. Ed. (1980), Shire S., Monoclonal Antibodies: Meeting the Challenges in Manufacturing, Formulation, Delivery and Stability of Final Drug Product, 1st Ed., Woodhead Publishing (2015), § 4 and Falconer R. J., Biotechnology Advances (2019), 37, 107412.

Exemplary pharmaceutical compositions comprising vixarelimab or a variant thereof are lyophilized, aqueous, frozen, etc. An exemplary pharmaceutical composition comprises 180 mg/mL of vixarelimab, 25 mM arginine hydrochloride, 20 mM histidine, 125 mM sodium chloride, 0.05% (w/v) polysorbate 80 (PS80), at approximately pH 6.6. Another exemplary pharmaceutical composition comprises 200 mg/mL of vixarelimab, 25 mM arginine hydrochloride, 20 mM histidine, 125 mM sodium chloride, 0.05% (w/v) polysorbate 80 (PS80), at approximately pH 6.6.

The pharmaceutical composition herein may also contain more than one active ingredients, as necessary, for the particular indication being treated, preferably those with complementary activities that do not adversely affect each other. For example, it may be desirable to further provide an aminosalicylate, a corticosteroid, a biologic, a thiopurine, methotrexate, a calcineurin inhibitor, e.g., cyclosporine or tacrolimus, and/or an antibiotic. Examples of aminosalicylates include 5-aminosalicylic acid, sulfasalazine, balsalazide, olsalazine and mesalazine, in forms like Eudragit-S-coated, pH-dependent mesalamine, ethylcellulose-coated mesalamine, and multimatrix-release mesalamine.

Examples of a steroid include corticosteroids or glucocorticosteroids. Examples of a corticosteroid include prednisone and hydrocortisone or methylprednisolone, or a second generation corticosteroid, e.g., budesonide or azathioprine; e.g., in forms like a hydrocortisone enema or a hydrocortisone foam.

Examples of biologics include etanercept; an antibody to tumor necrosis factor alpha, e.g., infliximab, adalimumab or certolizumab; an antibody to IL-12 and IL-23, e.g., ustekinumab; vedolizumab; etrolizumab, and natalizumab.

Examples of thiopurines include azathioprine (AZA), 6-mercaptopurine and thioguanine.

Examples of antibiotics include vancomycin, rifaximin, metronidazole, trimethoprim, sulfamethoxazole, diaminodiphenyl sulfone and ciprofloxacin; and antiviral agents like ganciclovir.

Such active ingredients are suitably present in combination in amounts that are effective for the purpose intended.

The pharmaceutical compositions to be used for in vivo administration are generally sterile. Sterility may be readily accomplished, e.g., by filtration through sterile filtration membranes

### Therapeutic Methods of Routes of Administration

Any of vixarelimab or a variant thereof may be used in therapeutic methods.

In one aspect, vixarelimab or a variant thereof for use as a medicament is provided. In further aspects, vixarelimab or a variant thereof for use in treating a gastrointestinal inflammatory condition. In some embodiments, the gastrointestinal inflammatory condition is IBD (inflammatory bowel disease). In some embodiments the inflammatory bowel disease is ulcerative colitis (UC) or Crohn's disease (CD). In some embodiments, the gastrointestinal inflammatory condition is colitis (e.g., colitis caused by environmental insults (e.g., caused by or associated with a therapeutic regimen, such as chemotherapy, radiation therapy, etc.), infectious colitis, ischemic colitis, collagenous or lymphocytic colitis, necrotizing enterocolitis, colitis in conditions such as chronic granulomatous disease or celiac disease, food allergies, gastritis, gastroenteritis, infectious gastritis or enterocolitis (e.g., Helicobacter pylori-infected chronic active gastritis), and other forms of gastrointestinal inflammation caused by an infectious agent, or indeterminate colitis.

In some embodiments, the gastrointestinal inflammatory condition is ulcerative colitis (UC) or Crohn's disease (CD).

In some embodiments, the gastrointestinal inflammatory condition is active moderate-to-severe ulcerative colitis. In some embodiments, the ulcerative colitis is mild to moderate distal colitis. In some embodiments, the ulcerative colitis is mild to moderate extensive colitis. In some embodiments, the ulcerative colitis is severe colitis.

In some embodiments, the gastrointestinal inflammatory condition is Crohn's disease (CD). In some embodiments, the Crohn's disease is in acute disease stage. In some embodiments, the Crohn's disease is in induced clinical remission stage. In some embodiments, the Crohn's disease is in a sustained response/remission stage.

In some embodiments, the Crohn's disease is mild to moderate disease. In some embodiments, the Crohn's disease is moderate to severe disease. In some embodiments, the Crohn's disease is severe/fulminant disease. In some embodiments, the Crohn's disease is ileal, ileocolonic, or colonic disease.

In some embodiments of any of the methods, the method further comprises administering one or more active ingredients selected from an aminosalicylate; a steroid; a biologic; a thiopurine; methotrexate; a calcineurin inhibitor, e.g., cyclosporine or tacrolimus; and an antibiotic.

In some embodiments of any of the methods, the method comprises administering the further active ingredient in an oral or topical formulation.

Examples of aminosalicylates include 5-aminosalicylic acid, sulfasalazine, balsalazide, olsalazine and mesalazine, in forms like Eudragit-S-coated, pH-dependent mesalamine, ethylcellulose-coated mesalamine, and multimatrix-release mesalamine.

Examples of a steroid include corticosteroids or glucocorticosteroids. Examples of a corticosteroid include prednisone and hydrocortisone or methylprednisolone, or a second generation corticosteroid, e.g., budesonide or azathioprine; e.g., in forms like a hydrocortisone enema or a hydrocortisone foam.

Examples of biologics include etanercept; an antibody to tumor necrosis factor alpha, e.g., infliximab, adalimumab or certolizumab; an antibody to IL 12 and IL23, e.g., ustekinumab; vedolizumab; etrolizumab, and natalizumab.

Examples of thiopurines include azathioprine, 6-mercaptopurine and thioguanine.

Examples of antibiotics include vancomycin, rifaximin, metronidazole, trimethoprim, sulfamethoxazole, diaminodiphenyl sulfone and ciprofloxacin; and antiviral agents like ganciclovir.

In further aspects, the present disclosure relates to vixarelimab or a variant thereof for use in reducing inflammation in the gastrointestinal tract in a subject comprising administering to the subject an effective amount of vixarelimab or a variant thereof to reduce inflammation in the gastrointestinal tract. The subject according to any of the above aspects is preferably a human.

In a further aspect, the present disclosure relates to the use of vixarelimab or a variant thereof in the manufacture or preparation of a medicament. In some embodiments, the medicament is for treatment of a gastrointestinal inflammatory disorder. In a preferred embodiment, the gastrointestinal inflammatory disorder is an inflammatory bowel disease, such as ulcerative colitis or Crohn's disease. In some embodiments, the medicament is for facilitating remission in a subject such as clinical remission, endoscopic remission, radiographic remission, histological remission, surgical remission, and/or biochemical remission. In a further aspect, the medicament is for use in a method of treating a gastrointestinal inflammatory disorder comprising administering to a subject having the gastrointestinal inflammatory disorder an effective amount of the medicament. In some embodiments, the method further comprises administering to the subject an effective amount of at least one additional therapeutic agent, e.g., as described below. In a further aspect, the medicament is for reducing inflammation in the gastrointestinal tract of the individual. In a further aspect, the medicament is for use in a method of reducing symptoms of an inflammatory bowel disease such as diarrhea, fever, fatigue, abdominal pain, cramping, hematochezia, reduced appetite, and/or unintended weight loss) in a subject comprising administering to the subject an effective amount of the medicament to reduce one or more of these symptoms. The subject according to any of the above aspects may be a human.

In a further aspect, a method for treating an IBD is provided. In some embodiments of any of the above aspects, the IBD is ulcerative colitis. In some embodiments of any of the above aspects, the ulcerative colitis is active moderate to severe ulcerative colitis. In some embodiments, the method comprises administering to a subject having such IBD an effective amount of vixarelimab or variant thereof. In some embodiments, the method further comprises administering to the subject an effective amount of at least one additional therapeutic agent, as described below.

In a further aspect, a method for causing remission in a subject having an IBD, wherein the remission includes clinical remission, endoscopic remission, radiographic remission, histological remission, surgical remission, and/or biochemical remission in the subject is provided. In some embodiments of any of the above aspects, the IBD is ulcerative colitis. In some embodiments of any of the above aspects, the ulcerative colitis is active moderate to severe ulcerative colitis. In some embodiments, the method comprises administering to the subject an effective amount of vixarelimab or variant thereof to cause the remission.

In a further aspect, a pharmaceutical composition comprising any of vixarelimab or a variant thereof, e.g., for use in any of the above therapeutic methods is provided. In some embodiments, the pharmaceutical composition comprises any of vixarelimab or a variant thereof and a pharmaceutically acceptable carrier. In some embodiments, a pharmaceutical composition comprises any of vixarelimab or a variant thereof and at least one additional therapeutic agent, e.g., as described below.

Antibodies of this disclosure can be administered alone or used in a combination therapy. For instance, the combination therapy includes administering vixarelimab or a variant thereof and administering at least one additional therapeutic agent (e.g., one, two, three, four, five, or six additional therapeutic agents). In some embodiments, the combination therapy comprises administering vixarelimab or a variant thereof and administering at least one additional IBD therapeutic agent selected from an aminosalicylate, an immunomodulatory agent, a tumor necrosis factor (TNF) antagonist, an anti-integrin agent, a mucosal addressin cell adhesion molecule (MAdCAM) antagonist, an IL-23 antagonist, an IL-12 antagonist, an IL-12/IL-23 antagonist, an antibiotic, or a corticosteroid. In some embodiments, the additional IBD therapy is an aminosalicylate. In some embodiments, the aminosalicylate comprises 5-aminosalicylic acid (5-ASA). In some embodiments, the additional IBD therapy is an immunomodulatory agent (e.g., azathioprine, mercaptopurine, cyclosporine, tacrolimus, sirolimus, mycophenolic acid, or methotrexate). In some embodiments, the additional IBD therapy is a TNF antagonist. In some embodiments, the TNF antagonist is an anti-TNF antibody (e.g., infliximab, adalimumab, golimumab, certolizumab pegol, a fragment thereof, or a derivative or biosimilar thereof) or a soluble TNF receptor (e.g., etanercept, a fragment thereof, or a derivative or biosimilar thereof). In some embodiments, the additional IBD therapy is an anti-integrin agent. In some embodiments, the anti-integrin agent is an anti-integrin antibody (e.g., an anti-α4-integrin antibody (e.g., natalizumab, vedolizumab, a fragment thereof, or a derivative thereof)). In some embodiments, the additional IBD therapy is a MAdCAM antagonist. In some embodiments, the MAdCAM antagonist is an anti-MAdCAM antibody (e.g. PF-00547659 or SHP647). In some embodiments, the additional IBD therapy is an IL-23 antagonist. In some embodiments the IL-23 antagonist is an anti-IL-23 antibody (e.g., briakizumab, guselkumab, risankizumab, tildrakizumab, or ustekinumab). In some embodiments, the additional IBD therapy is an IL-12 antagonist. In some embodiments, the IL-12 antagonist is an anti-IL-12 antibody (e.g., ABT-874/J695). In some embodiments, the additional IBD therapy is an IL-12/IL-23 antagonist. In some embodiments, the IL-12/IL-23 antagonist is an anti-IL-12/IL-23 antibody (e.g., ustekinumab or briakinumab).

Such combination therapies noted above encompass combined administration (where two or more therapeutic agents are included in the same or separate pharmaceutical compositions), and separate administration, in which case, administration of the antibody of this disclosure can occur prior to, simultaneously, and/or following, administration of the additional therapeutic agent or agents. In some embodiments, administration of vixarelimab or a variant thereof and administration of an additional therapeutic agent occur within about one month, or within about one, two or three weeks, or within about one, two, three, four, five, or six days, of each other. In some embodiments, vixarelimab and the additional therapeutic agent are administered to the patient on Day 1 of the treatment.

An antibody of this disclosure (and any additional therapeutic agent) can be administered by any suitable means, including parenteral, intrapulmonary, and intranasal, and, if desired for local treatment, intralesional administration. Parenteral infusions include subcutaneous, intramuscular, intravenous, intraarterial, or intraperitoneal administration. Dosing can be by any suitable route, e.g., by injections, such as subcutaneous or intravenous injections, depending in part on whether the administration is brief or chronic. Various dosing schedules including but not limited to single or multiple administrations over various time-points, bolus administration, and pulse infusion are contemplated herein.

Vixarelimab or a variant thereof would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally, formulated with one or more agents currently used to prevent or treat the disorder in question. The effective amount of such other agents depends on the amount of antibody present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate. Optionally, vixarelimab can be formulated in a buffer comprising 20 mM L-histidine, 25 mM L-arginine hydrochloride, 125 mM sodium chloride, 0.05% (w/v) polysorbate 80, pH 6.6-6.8 and diluted to 180 mg/mL by formulation buffer.

For the prevention or treatment of disease, the appropriate dosage of an antibody of this disclosure (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. For repeated administrations over several days or longer, depending on the condition, the treatment would generally be sustained until a desired suppression of disease symptoms occurs. The progress of this therapy is easily monitored by conventional techniques and assays.

In some embodiments of any of the above aspects, the therapeutically effective dose is about 720 mg of the anti-OSMRβ antibody (e.g., vixarelimab). In some embodiments of any of the above aspects, the therapeutically effective dose is 720 mg of the anti-OSMRβ antibody (e.g., vixarelimab). In some embodiments of any of the above aspects, the anti-OSMRβ antibody (e.g., vixarelimab) is administered for at least 12 weeks. In some embodiments of any of the above aspects, the therapeutically effective dose is about 720 mg of the anti-OSMRβ antibody (e.g., vixarelimab) for at least 12 weeks. In some embodiments of any of the above aspects, the therapeutically effective dose is 720 mg of the anti-OSMRβ antibody (e.g., vixarelimab) for at least 12 weeks. In some embodiments of any of the above aspects, the anti-OSMRβ antibody (e.g., vixarelimab) is administered at weeks 0, 1, 2, and every 2 weeks (Q2W) thereafter. In some embodiments of any of the above aspects, the therapeutically effective dose is administered at weeks 0, 1, 4, and every 4 weeks (Q4W) thereafter. In some embodiments of any of the above aspects, the anti-OSMRβ antibody (e.g., vixarelimab) is administered at week 0. In some embodiments, the anti-OSMRβ antibody (e.g., vixarelimab) is administered at week 1. In some embodiments of any of the above aspects, the anti-OSMRβ antibody (e.g., vixarelimab) is administered at week 2. In some embodiments of any of the above aspects, the anti-OSMRβ antibody (e.g., vixarelimab) is administered at week 4. In some embodiments of any of the above aspects, the anti-OSMRβ antibody (e.g., vixarelimab) is administered every 2 weeks (Q2W). In some embodiments of any of the above aspects, the anti-OSMRβ antibody is administered every 4 weeks (Q4W). In some embodiments of any of the above aspects, the anti-OSMRβ antibody (e.g., vixarelimab) is administered for 48 weeks. In some embodiments of any of the above aspects, the anti-OSMRβ antibody (e.g., vixarelimab) is administered for 12 to 48 weeks. In some embodiments of any of the above aspects, the anti-OSMRβ antibody (e.g., vixarelimab) is administered intravenously. In some embodiments of any of the above aspects, the anti-OSMRβ antibody (e.g., vixarelimab) is administered subcutaneously.

In some embodiments of any of the above aspects, the therapeutically effective dose of about 720 mg of the anti-OSMRβ antibody (e.g., vixarelimab) is subcutaneously administered. In some embodiments of any of the above aspects, the therapeutically effective dose of 720 mg of the anti-OSMRβ antibody (e.g., vixarelimab) is subcutaneously administered. In some embodiments of any of the above aspects, the anti-OSMRβ antibody (e.g., vixarelimab) is subcutaneously administered for at least 12 weeks. In some embodiments of any of the above aspects, the therapeutically effective dose of about 720 mg of the anti-OSMRβ antibody (e.g., vixarelimab) is subcutaneously administered for at least 12 weeks. In some embodiments of any of the above aspects, the therapeutically effective dose of 720 mg of the anti-OSMRβ antibody (e.g., vixarelimab) is subcutaneously administered for at least 12 weeks. In some embodiments of any of the above aspects, the anti-OSMRβ antibody (e.g., vixarelimab) is subcutaneously administered at weeks 0, 1, 2, and every 2 weeks (Q2W) thereafter. In some embodiments of any of the above aspects, the therapeutically effective dose is subcutaneously administered at weeks 0, 1, 4, and every 4 weeks (Q4W) thereafter. In some embodiments of any of the above aspects, the anti-OSMRβ antibody (e.g., vixarelimab) is subcutaneously administered at week 0. In some embodiments, the anti-OSMRβ antibody (e.g., vixarelimab) is subcutaneously administered at week 1. In some embodiments of any of the above aspects, the anti-OSMRβ antibody (e.g., vixarelimab) is subcutaneously administered at week 2. In some embodiments of any of the above aspects, the anti-OSMRβ antibody (e.g., vixarelimab) is subcutaneously administered at week 4. In some embodiments of any of the above aspects, the anti-OSMRβ antibody (e.g., vixarelimab) is subcutaneously administered every 2 weeks (Q2W). In some embodiments of any of the above aspects, the anti-OSMRβ antibody (e.g., vixarelimab) is subcutaneously administered every 4 weeks (Q4W). In some embodiments of any of the above aspects, the anti-OSMRβ antibody (e.g., vixarelimab) is administered for 48 weeks. In some embodiments of any of the above aspects, the anti-OSMRβ antibody (e.g., vixarelimab) is administered for 12 to 48 weeks.

### Articles of Manufacture

In another aspect of this disclosure, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the present disclosure. The label or package insert indicates that the composition is used for treating the condition of choice. Moreover, the article of manufacture may comprise (a) a first container with a composition contained therein, wherein the composition comprises an antibody of the present disclosure; and (b) a second container with a composition contained therein, wherein the composition comprises a further cytotoxic or otherwise therapeutic agent. The article of manufacture in this aspect of the present disclosure may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Examples

The disclosure will be further understood by reference to the following examples, which are intended to be purely exemplary of the disclosure herein. The present disclosure is not limited in scope by the exemplified embodiments, which are intended as illustrations of single aspects of the disclosure only.

### Example 1. Selecting a Dosing Regimens for IBD

The target efficacious concentration (C_{eff}) was estimated based on the combination of nonclinical data from cynomolgus monkey itch studies and clinical data from a phase 1 clinical study in subjects diagnosed with atopic dermatitis (AD) and adjusted based on the in vitro potency differences between human skin keratinocytes and intestinal fibroblasts to generate an estimated adjusted C_{eff} (C_{eff,adj}) for IBD.

In a cynomolgus monkey itch study, vixarelimab was used to inhibit scratching behavior, interpreted as a sign of pruritus, induced by a single intradermal administration of recombinant human (rh) IL-31. Supraphysiologic intradermal challenge doses of rhIL-31 between 3 and 24 μg/kg were tested and all induced scratching, with 3 μg/kg resulting in a robust response with minimal variability. Administration of a single IV vixarelimab dose (1, 3, or 10 mg/kg) resulted in dose- and time-dependent reduction in rhIL-31-induced scratching. Higher serum concentrations of vixarelimab resulted in longer scratching inhibition, and duration of effect helped establish 5-8 μg/mL as the serum concentration threshold for vixarelimab efficacy in this model system.

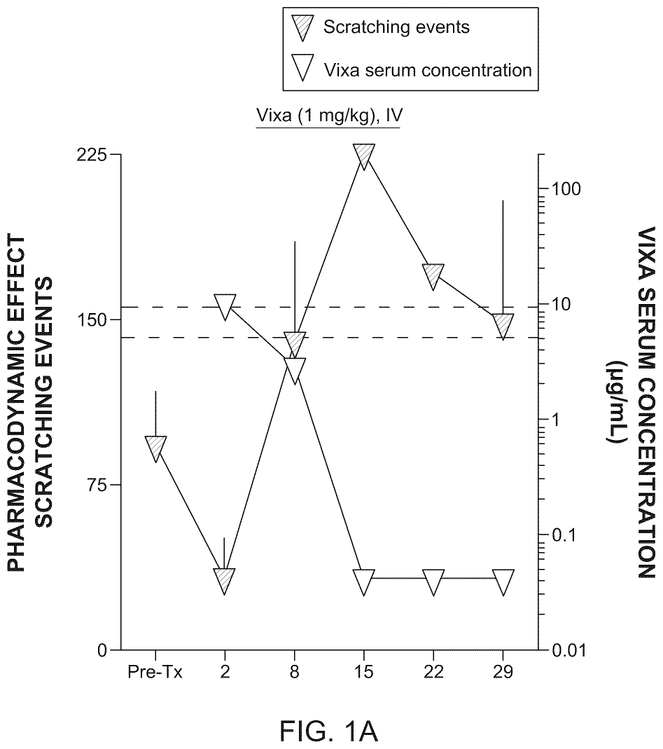

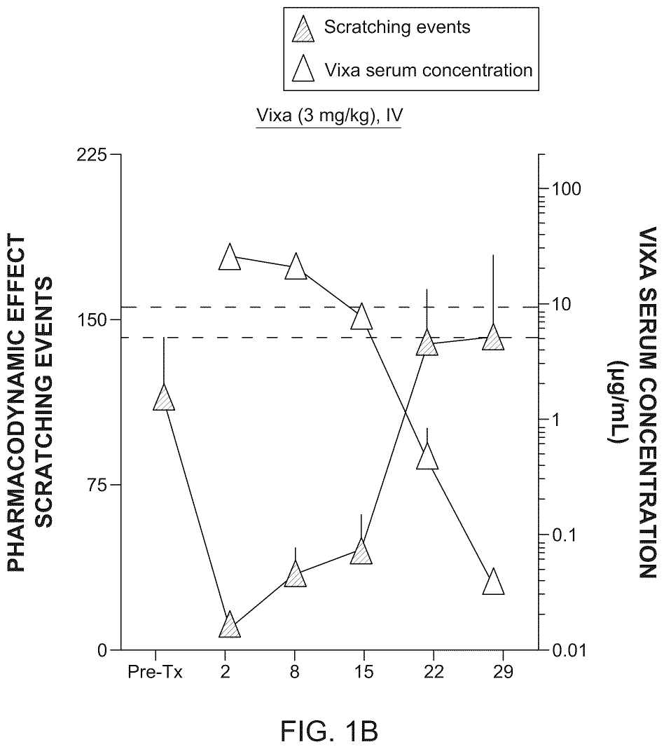

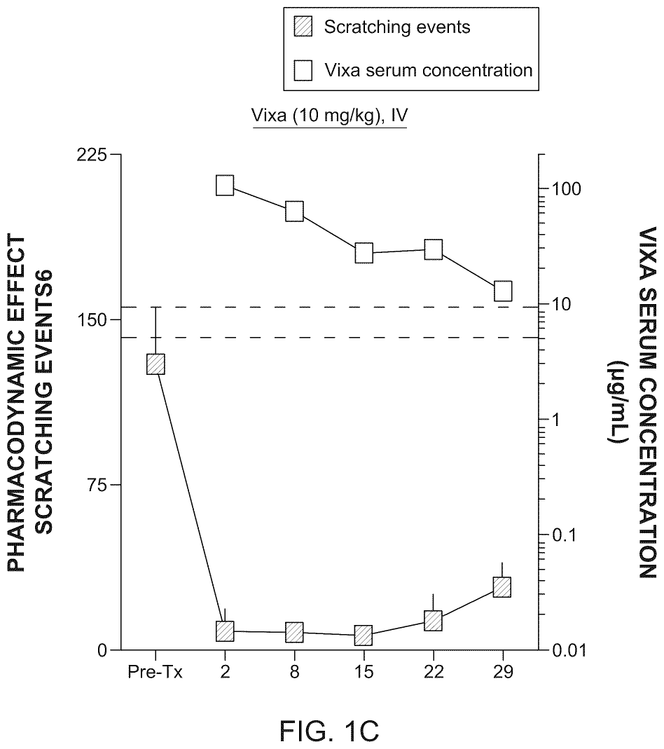

Repeated subcutaneous administration of vixarelimab, 1 mg/kg weekly, 3 mg/kg biweekly, or 8 mg/kg monthly, demonstrated prolonged and significant reduction of IL-31-induced scratching behavior. Monkeys were challenged with rhIL-31 by intradermal injection at various time points following vixarelimab injection. Scratching events following hIL-31 challenge were reported for each group. Concentrations of vixarelimab at the same dosing regimens were simulated and correlated with reduction in rhIL-31-induced scratching, verifying a C_{eff} threshold of 5-8 μg/mL for inhibiting pruritic responses in this model. (See FIG. 1A to FIG. 1C).

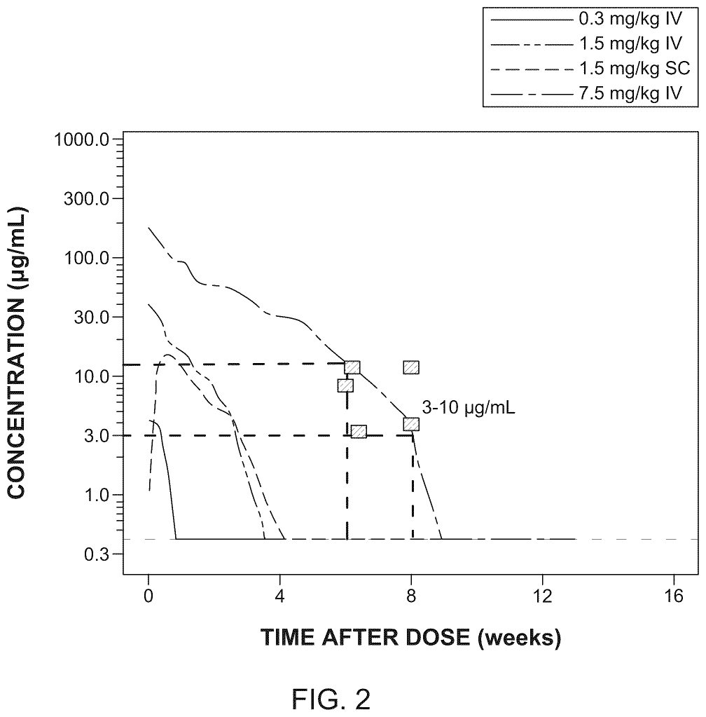

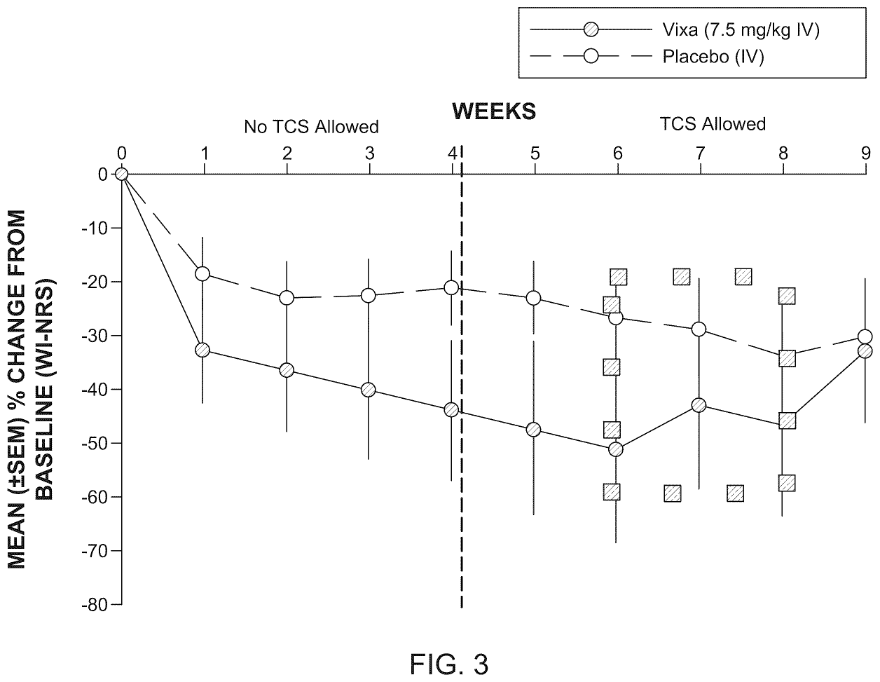

The human Phase 1b clinical study of vixarelimab treatment involved IV administration of vixarelimab at doses of 0.3 mg/kg, 1.5 mg/kg, or 7.5 mg/kg, 10 mg/kg or 20 mg/kg and SC administration at doses of 1.5 mg/kg or 360 mg. Patients were monitored for safety as well as for disease severity, pruritis intensity, and quality of life measures including sleep quality. It was found that a sustained efficacy lasted 6-8 weeks following a single IV dose of 7.5 mg/kg in patients with AD, supporting the C_{eff} of 5-8 μg/mL that was identified in the cynomolgus study above. (See FIGS. 2 and 3)

Independently, an in vitro potency study was performed in order to compare the ability of vixarelimab to inhibit OSM-induced STAT3 phosphorylation in human primary IBD-derived intestinal fibroblasts and normal keratinocytes. This study allowed use of the C_{eff} determined in the cynomolgus and human studies above to be converted into an estimated adjusted C_{eff} (C_{eff,adj}) for use in a phase 2 study of vixarelimab in IBD.

Four healthy donor-derived human keratinocytes (used as assay control) were purchased from Lonza (Basel, Switzerland). Twelve patient-derived primary intestinal fibroblasts with IBD or other intestinal diseases were from Genentech. All primary cells were cultured in complete media containing RPMI-1640 with 10% heat-inactivated fetal bovine serum, 2 mM L-glutamine, and 1% penicillin-streptomycin. The cells were seeded at 20,000 cells per well into a 96-well plate (Product No. 3595; Corning; Corning, NY) and incubated overnight in an incubator at 37° C. with 5% CO₂. The next day, vixarelimab antibody was serially diluted 3-fold from a starting concentration of 100 μg/mL for a total of 10 dilutions in RPMI-1640 complete media. To examine the inhibitory potency of vixarelimab against different levels of OSM, patient-derived primary intestinal fibroblasts were treated with either 1 or 10 ng/ml (final concentration) of OSM (Purification Batch PURIBY00559; Genentech) in the presence of antibodies, and normal human primary keratinocyte cells were treated with 10 ng/ml OSM in the presence of antibodies. Forty microliters of serially diluted vixarelimab antibody and 40 μL OSM were mixed and incubated for 10 minutes at room temperature. Then 50 μL of the mixture was added to each well of the cell plate. The cell plate was incubated at 37° C. for 15 minutes. After this incubation, phosphorylated STAT3 (pSTAT3) was measured using the Phospo-STAT3 (Tyr705) kit (Catalog No. K150SVD-4; Meso Scale Discovery [MSD]; Gaithersburg, MD) according to the following steps. Cell culture media were removed from the plate, and 60 μL of lysis buffer containing phosphatase and protease inhibitors were added to each well to lyse the cells. After 1 hour of incubation at 4° C., 25 L of cell lysates were transferred using a Biomek i5 Automated Workstation (Beckman Coulter; Indianapolis, IN) to an MSD plate that was previously blocked and washed. The cell lysates were incubated at 4° C. overnight on a shaker. Then the plate was washed 3 times with 200 μL per well of Tris buffer, followed by the addition of 25 μL SULFO-TAG™-labeled anti-phospho-STAT3 detection antibody into each well. After 1 hour of incubation at room temperature on a shaker, the plate was washed 3 times with 200 μL Tris buffer per well, and 150 μL surfactant-based read buffer was added to each well before the plate was read on an MSD MESO SECTOR S 600 instrument.

The percentage of pSTAT3 for each treatment condition was calculated using the following equation, where the maximum is the MSD signal of OSM only and the minimum is the MSD signal of RPMI 1640 media only:

pSTAT3(% inhibition)=[1−(MSD signal−minimum)÷(maximum−minimum)]×100

The pSTAT3 (% inhibition) was plotted as a function of vixarelimab concentrations, and the data were fitted to a sigmoidal 4-parameter logistic (4PL) model using Prism (GraphPad; La Jolla, CA). The 50% inhibitory concentration (IC₅₀) value for each donor was determined as the concentration reaching 50% inhibition of maximum activity. The concentration that leads to 90% maximal inhibitory response (90% inhibitory concentration; IC₉₀) was calculated using modeled parameters.

The results show that vixarelimab consistently inhibits OSM-induced STAT3 phosphorylation across a panel of patient-derived primary intestinal fibroblasts. The mean and standard deviation of IC₅₀ and IC₉₀ values were determined by the concentration-response curve fitted to a sigmoidal 4PL model. The results are summarized in Table 3 below.

TABLE 3
Vixarelimab mean and standard deviation of IC50 and IC90

| | | Mean of | Standard | Mean of | Standard |
|---|---|---|---|---|---|
| | [OSM] | IC₅₀ | deviation of | IC₉₀ | deviation of |
| Cell Type | (ng/mL) | (nM) | IC₅₀ | (nM) | IC₉₀ |
| IBD-derived | 1 | 0.14 | 0.05 | 0.99 | 0.37 |
| intestinal | | | | | |
| Fibroblasts | | | | | |
| IBD-derived | 10 | 1.15 | 0.36 | 8.91 | 1.60 |
| intestinal | | | | | |
| Fibroblasts | | | | | |
| Normal | 10 | 0.36 | 0.18 | 8.32 | 2.63 |
| Keratinocytes | | | | | |

The results of the in vitro study were used to convert the C_{eff} of 8 μg/mL to a range of C_{eff,adj} between 3-25 μg/mL for IBD, using a difference in in vitro IC₅₀ for OSM-induced pSTAT3 activation between keratinocytes and intestinal fibroblasts.

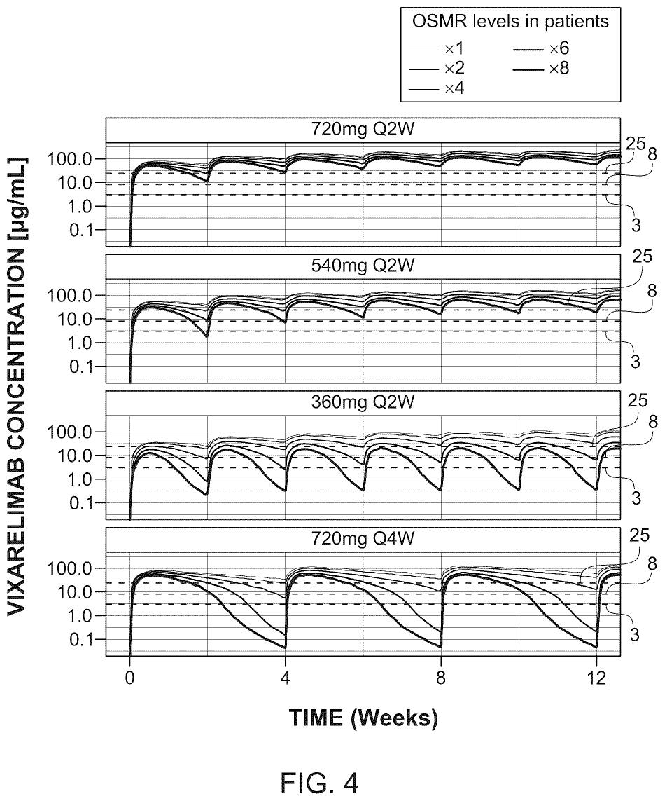

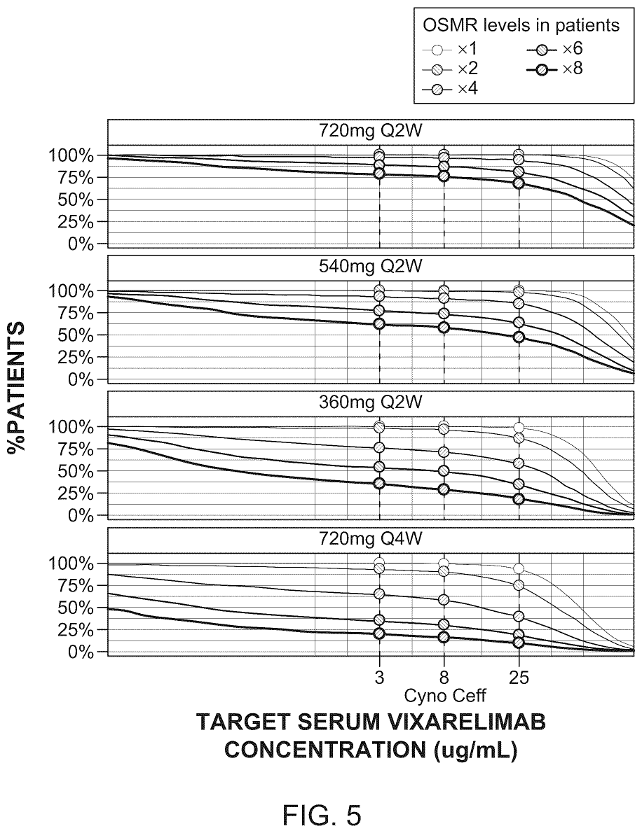

Next, the PK profile of vixarelimab was simulated to estimate the steady-state trough concentration (C_{min,ss}) coverage of the estimated C_{eff,adj} at various subcutaneous (SC) dosing regimens with area under the concentration-time curve (AUC) at or lower than that of the highest clinically tested dose in the multiple-dose setting, 360 mg QW SC. Simulation was performed with a preliminary target-mediated drug disposition (TMDD) population PK model developed with available clinical PK data from healthy subjects and AD/PN patients (Studies KPL-716-C001, Phase 2a portion of KPL-716-C201 and KPL-716-C202). The PK profiles across healthy subjects and AD/PN patient populations appeared comparable; however, the simulation incorporated assumptions of varying degrees of OSMR overexpression in IBD patients compared with healthy subjects ranging up to 8-fold systemic OSMR expression in IBD patients, corresponding to a reported mean of up to 8-fold increase in OSMR mRNA overexpression in IBD intestinal mucosa compared to controls (West et al., 2017, ibid). However, as the systemic OSMR levels rather the local OSMR levels in the intestinal tissue are represented in the population PK model, the upper end of the OSMR level assumptions in the PK simulations is considered an extreme scenario. The PK profiles are illustrated in FIGS. 4 and 5.

PK simulations showed that following administration of 720 mg Q2W, >90% of IBD patients are predicted to have C_{min,ss} above the estimated upper end of C_{eff,adj} (25 μg/mL) at up to 4-fold systemic OSMR level increase; even at the extreme scenario of 8-fold systemic OSMR level increase, the majority (˜70%) of IBD patients are predicted to have C_{min,ss} above 25 μg/mL. In contrast, lower Q2W doses, such as 540 mg Q2W and 360 mg Q2W, are predicted to have less consistent coverage of C_{eff,adj} especially at high OSMR levels.

The model-predicted steady-state AUC of 720 mg Q2W is expected to be the same as steady-state AUC of 360 mg QW. The model-predicted steady-state maximum serum concentration (Cₘₐₓ) of 720 mg Q2W is slightly higher than the steady-state Cₘₐₓ of 360 mg QW; however, this small difference is expected to fall well within the expected interindividual pharmacokinetic (PK) variability. In addition, a 720 mg SC dose was used as a loading dose in the Phase IIa portion of Study KPL-716-C201 and Study KPL-716-C202 and was well tolerated.

The selection of 720 mg Q2W is also supported by the available nonclinical toxicology data, with an AUC- and Cₘₐₓ-based safety margin of >50 based on the no-observed-adverse-effect level from the 26-week chronic toxicology study in cynomolgus monkeys. Overall, the predicted satisfactory coverage of C_{eff,adj} across a wide range of OSMR levels and evidence on safety support the selection of 720 mg Q2W as the top dose in the Phase 2 study of vixarelimab in IBD.

If clinical activity is observed with 720 mg Q2W, it is important to understand whether a more patient-friendly dosing regimen with a more extended dosing interval, 720 mg Q4W, would also be efficacious. Based on the PK simulations, at 720 mg Q4W, >90% and >70% of IBD patients are predicted to have C_{min,ss} above 25 μg/mL at 1-fold and 2-fold OSMR expression level, respectively. The dosing regimen of 720 mg Q4W is predicted to have approximately 2-fold lower AUC and 3-fold lower Cₘᵢₙ than 720 mg Q2W when assuming the same target levels as in healthy volunteers. The relatively wide exposure range achieved by the two dosing regimens will provide data to support dose/exposure-response/safety analysis to guide dose selection for future clinical trials. Therefore, 720 mg Q4W was selected as the low dose option in the Phase 2 study of vixarelimab in IBD.

### Example 2. Phase 2 Study to Evaluate Efficacy, Safety, and PK in Ulcerative Colitis (UC)

A Phase II, multicenter study was designed to evaluate the efficacy, safety, and pharmacokinetics of vixarelimab in patients with active moderate to severe UC, including 1) patients who have demonstrated inadequate response to, loss of response to, or intolerance to prior advanced therapy which includes biologics and targeted small molecules (advanced failures), and 2) patients who have demonstrated inadequate response to, loss of response to, or intolerance to prior conventional therapies (corticosteroids and/or immunosuppressants) but have not failed advanced therapy (conventional failures). Patients will be enrolled in either a randomized, parallel-group, double-bind, placebo controlled, dose-ranging part, or in an open-label active treatment part.

The study consists of screening period of up to 35 days, a 48-week treatment period, and a safety follow-up period for 10 weeks following the final dose of study treatment. The induction period (Weeks 0-12) will test the induction of clinical remission. After completion of the induction period, all patients, irrespective of clinical response or remission, will be eligible to continue study treatment during an active treatment extension (ATE) period (Weeks 12-48).

During the active treatment extension (ATE) period, all patients will receive vixarelimab, including patients who received placebo during the induction period, and the durability of clinical response and remission will be explored.

TABLE 4
Clinical Trial Objectives and Corresponding Endpoints

| Primary Objective | Corresponding Endpoint |
|---|---|
| To evaluate the induction efficacy of | Clinical remission at Week 12, with clinical |
| vixarelimab compared with placebo | remission defined as mMS ^{a} of ≤2, |
| To evaluate the induction efficacy of | including: |
| vixarelimab compared with placebo in | Stool frequency subscore ≤ 1 |
| advanced failures | Rectal bleeding subscore= 0 |
| | Endoscopy subscore ≤ 1 (score of 1 modified |
| | to exclude friability) ^{b} |
| To evaluate the induction efficacy of | Clinical response at Week 12, with clinical |
| vixarelimab compared with placebo | response defined as meeting both of the |
| To evaluate the induction efficacy of | following criteria: |
| vixarelimab compared with placebo in | Decrease from baseline in the mMS ^{a} |
| advanced failures | of ≥ 2 and > 30% reduction from |
| | baseline |
| | Decrease in rectal bleeding subscore |
| | of ≥ 1 or absolute rectal bleeding subscore |
| | of ≤ 1 |
| | Endoscopic improvement at Week 12, with |
| | endoscopic improvement defined as a Mayo |
| | endoscopy subscore of ≤ 1 (score of 1 |
| | modified to exclude friability) ^{b} |
| | Endoscopic remission at Week 12, with |
| | endoscopic remission defined as a Mayo |
| | endoscopy subscore of 0 ^{b} |
| To evaluate the safety of vixarelimab compared | Incidence and severity of adverse events, |
| with placebo | with severity determined according to the |
| | DAIDS toxicity grading scale ^{c} |
| | Change from baseline in selected vital signs |
| | Change from baseline in selected clinical |
| | laboratory test results |
| To characterize the pharmacokinetics of | Serum concentration of vixarelimab at |
| vixarelimab | specified time points |
| To evaluate the immune response to | Prevalence of ADAs at baseline and incidence |
| vixarelimab | of ADAs during the study |
| To evaluate the induction efficacy of open-label | Clinical remission at Week 12 |
| vixarelimab | Clinical response at Week 12 |
| | Endoscopic improvement at Week 12 |
| | Endoscopic remission at Week 12 |
| To evaluate the long-term efficacy of | Sustained remission, defined as clinical |
| vixarelimab | remission at both Week 12 and Week 48 |
| | Clinical remission at Week 48 |
| | Clinical response at Week 48 |
| | Endoscopic improvement at Week 48 |
| | Endoscopic remission at Week 48 |
| | Change from baseline in UC bowel |
| | movement signs and symptoms at Week 12 |
| | and Week 48, as assessed by UC-PRO/SS |
| | score |
| | Improvement in UC bowel movement signs |
| | and symptoms at Week 12 and Week 48, as |
| | defined by the proportion of patients with |
| | a ≥ 6-point decrease in the UC-PRO/SS |
| | bowel domain score |
| | Change from baseline in UC functional |
| | signs and symptoms at Week 12 and Week |
| | 48, as assessed by UC-PRO/SS score |
| | Improvement in UC functional symptoms at |
| | Week 12 and Week 48, as defined by the |
| | proportion of patients with a ≥ 2-point |
| | decrease in the UC-PRO/SS functional |
| | domain score |
| To evaluate potential relationships between | Relationship between PK parameters for |
| vixarelimab exposure and the efficacy and | vixarelimab and efficacy endpoints |
| safety of vixarelimab | Relationship between PK parameters for |
| | vixarelimab and safety endpoints |
| To evaluate potential relationships between | Relationship between selected covariates |
| selected covariates and exposure to | and PK parameters for vixarelimab |
| vixarelimab | |
| To evaluate potential effects of ADAs | Relationship between ADA status and |
| | efficacy, safety, or PK endpoints |
| To identify and/or evaluate biomarkers that | Relationship between biomarkers in blood, |
| are predictive of response to vixarelimab | serum, stool, and colonic tissue, and |
| (i.e., predictive biomarkers), are associated | efficacy, safety, PK, immunogenicity, or |
| with progression to a more severe disease | other biomarker endpoints |
| state (i.e., prognostic biomarkers), are | |
| associated with acquired resistance to | |
| vixarelimab, are associated with susceptibility | |
| to developing adverse events or can lead to | |
| improved adverse event monitoring or | |
| investigation (i.e., safety biomarkers), can | |
| provide evidence of vixarelimab activity or | |
| disease improvement (i.e., PD biomarkers), | |
| can serve as a useful surrogate endpoint, or | |
| can increase the knowledge and | |
| understanding of disease biology and drug | |
| safety or pharmacokinetics | |
| To evaluate the use of automated assessment | Automated assessment of disease severity |
| of endoscopic videos to infer the efficacy of | Automated spatial assessment of mucosal |
| vixarelimab | features |

ADA = anti-drug antibody; DAIDS = Division of AIDS; mMS = modified Mayo Score; PD = pharmacodynamic; PK = pharmacokinetic; UC = ulcerative colitis; UC-PRO/SS = Ulcerative Colitis Patient-Reported Outcomes Signs and Symptoms.
^{a} Modified Mayo Score is the composite of three Mayo Score assessments: stool frequency, rectal bleeding, and centrally read endoscopy (see FIG. 7).
^{b} Endoscopy scores will be based on interpretation by a blinded central reader.
^{c} Adverse events will be graded according to the DAIDS Table for Grading the Severity of Adult and Pediatric Adverse Events (HHS 2017), with slight modifications for clarity and for alignment with internal practices.

The eligibility criteria for this study define patients with active moderate to severe UC with an mMS of 5-9 and endoscopic score of at least 2, consistent with regulatory guidance (U.S. Food and Drug Administration [FDA] 2022), who may benefit from the anticipated effects of vixarelimab. All patients considered for participation will have a diagnosis of moderate to severe UC established at least 3 months prior to screening, with active disease confirmed by clinical and endoscopic evidence during screening. Patients must have demonstrated an inadequate response, loss of response, or intolerance to prior conventional UC therapies and/or to up to 2 prior classes of approved advanced therapies (as defined below in “Inclusion Criteria for Advanced Failures”).

Patients will undergo an endoscopy with biopsy and a full Mayo Score assessment at screening (baseline), Week 12, and Week 48. Efficacy will be assessed using the modified Mayo Score (mMS), with the Mayo endoscopic subscore calculated on the basis of centrally read endoscopy. The mMS and partial Mayo Score (pMS) will be derived from the Mayo Score.

Patients who complete the treatment period (induction and optional ATE) will enter the safety follow-up period and undergo assessments at Week 52 and Week 56. Patients who discontinue study treatment without entering the optional ATE period will enter the safety follow-up period and undergo assessments at 6 weeks and 10 weeks following the final dose of study treatment. Patients who discontinue study treatment prematurely should return to the clinic for a treatment discontinuation visit within 14 days of the event and will then enter the safety follow-up period.

The total duration of study participation for each patient is expected to be approximately 25 weeks for patients who complete the induction treatment period only, and 61 weeks for patients who complete the optional ATE.

### Randomized Treatment Part

Approximately 210 patients, including advanced failures and conventional failures as defined below, will be enrolled in the randomized, double-blind treatment part of the study. The number of conventional failures will not exceed 40% of enrolled patients in the randomized part. Randomization will be stratified by previous advanced or conventional therapy failure and baseline modified Mayo Score (mMS).

Eligible patients will be randomized in a 1:1:1 ratio to one of the following treatment arms for the induction period (Weeks 0-12):
Vixarelimab 720 mg Q2W (720 mg at Weeks 0, 1, 2, and Q2W thereafter).
Vixarelimab 720 mg Q4W arm (720 mg at Weeks 0, 1, 4, and Q4W thereafter; placebo at Week 2 and Q4W thereafter).
Placebo Q2W arm (placebo at Weeks 0, 1, 2, and Q2W thereafter).

All patients who complete the induction period can receive SC injections of vixarelimab 720 mg during the optional ATE period (Weeks 12-48). Patients assigned to the vixarelimab 720 mg Q2W arm or vixarelimab 720 mg Q4W arm during the induction period will continue on their randomized treatment assignment during the optional ATE period:
Patients in the vixarelimab 720 mg Q2W arm will receive vixarelimab 720 mg Q2W.
Patients in the vixarelimab Q4W arm will receive vixarelimab 720 mg Q4W (starting at Week 12 and Q4W thereafter) and placebo Q4W (starting at Week 14 and Q4W thereafter).
Patients in the placebo arm during the induction period will receive vixarelimab 720 mg Q2W during the optional ATE period.

### Open-Label Treatment Part

Up to approximately 50 advanced failure patients, including 10 patients currently receiving anti-TNF therapy, will be enrolled in a separate open-label treatment part of the study. Patients receiving exclusionary medications prior to screening will complete a medication washout, including anti-TNF therapy, except for the 10 patients who will continue treatment with anti-TNF therapy during the induction period.

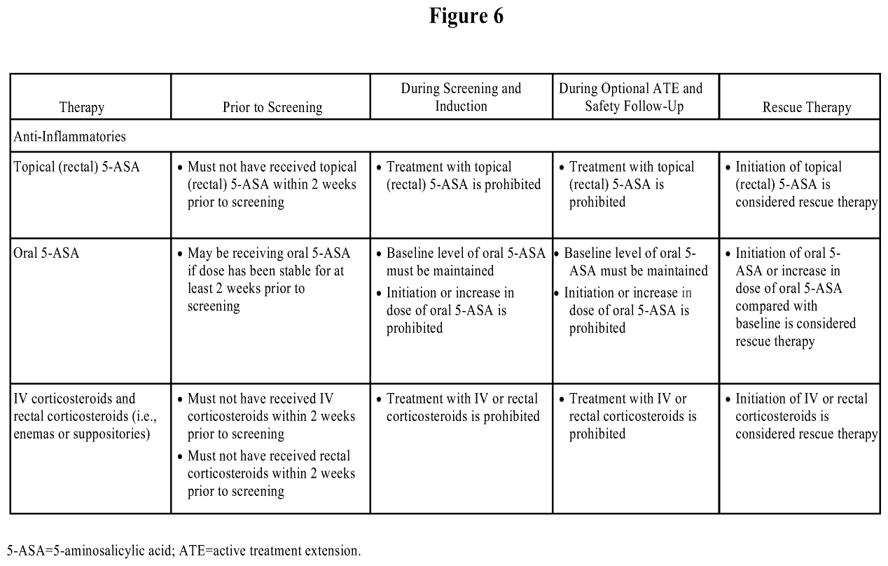

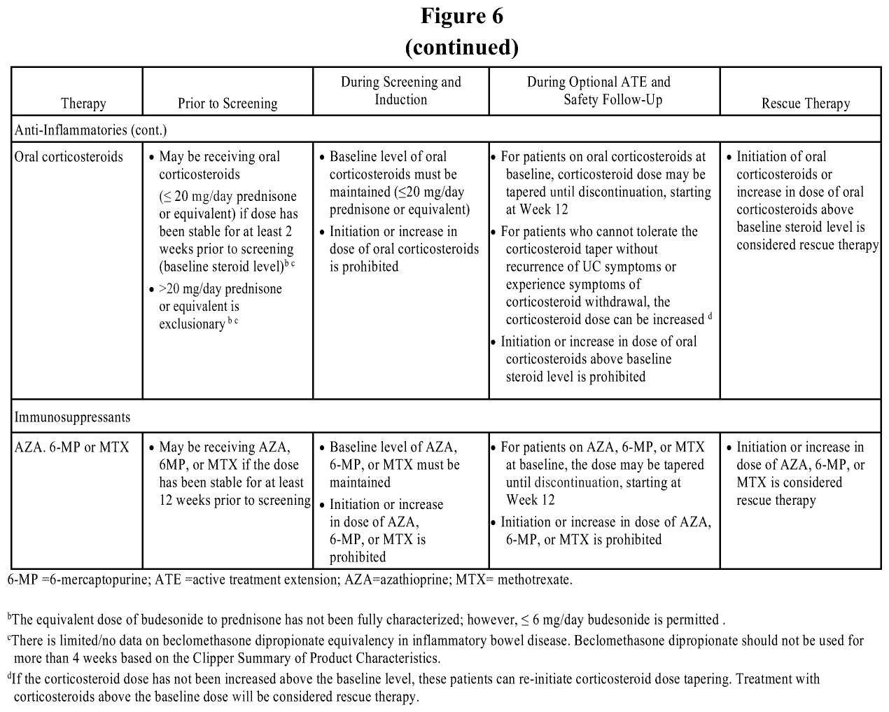

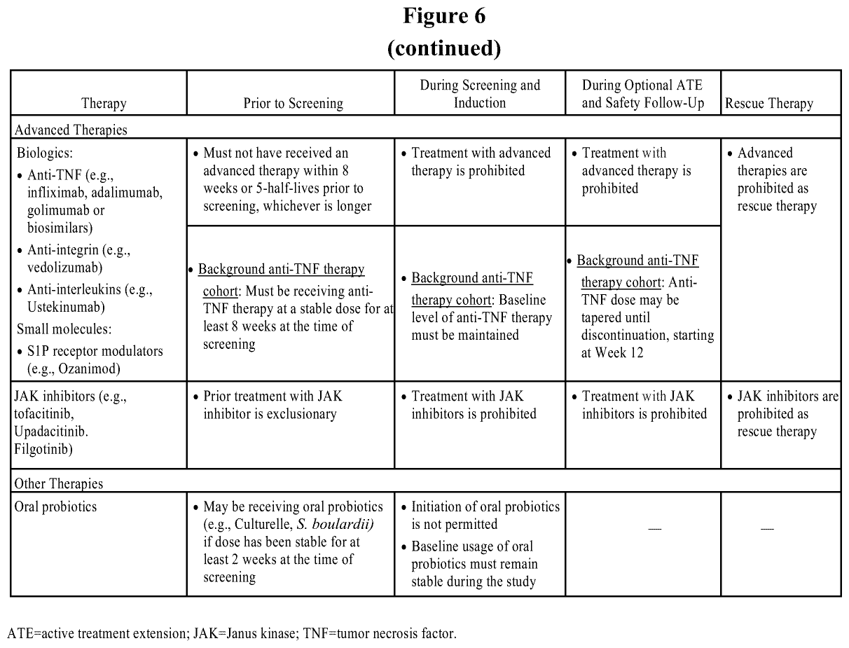

All patients in the open-label treatment part will be assigned to the vixarelimab 720 mg Q2W arm to receive SC injections of vixarelimab 720 mg at Week 0, 1, 2, and Q2W thereafter during the induction period (Weeks 0-12) and continue with 720 mg Q2W during the optional ATE period (Weeks 12-48), followed by follow-up visits approximately 6 weeks and 10 weeks after the final dose; these patients will undergo the same schedule of assessments as patients in the randomized treatment part. Restrictions regarding treatment with concomitant therapy are outlined in FIG. 6.

The end of this study is defined as the date when the last patient has completed his or her last visit or the date at which the last data point required for statistical analysis or safety follow-up is received from the last patient, whichever occurs later. The end of the study is expected to occur 56 weeks after the last patient is enrolled. The total duration of study participation for each patient is expected to be approximately 25 weeks for patients who complete the induction treatment period only, and 61 weeks for patients who complete the optional ATE.

### General Inclusion Criteria for All Patients

Inclusion criteria for study entry include:
≥18 years of age at time of signing Informed Consent Form
Ability to comply with the study protocol
Diagnosis of UC established at least 3 months prior to screening, confirmed by clinical and endoscopic evidence during screening. The diagnosis of UC should be corroborated by histopathology and documented by a histopathology report. A histopathologic examination should be performed at screening if no prior report is readily available.
Evidence of UC extending a minimum of 15 cm from the anal verge as determined by baseline endoscopy (flexible sigmoidoscopy or colonoscopy) performed during screening
Moderately to severely active UC, defined as a mMS of 5-9 with an endoscopic subscore of ≥2 (as determined by the central reading procedure described in Section 4.5.6) during the screening period, despite concurrent treatment
Any permitted background UC therapy must be at stable doses, as outlined below:
   May be receiving oral 5-ASA compounds at a dose that has been stable for ≥2 weeks immediately prior to screening.
   May be receiving oral corticosteroid therapy at a dose of ≤20 mg/day of prednisone (or equivalent) that has been stable for >2 weeks immediately prior to screening.
   May be receiving oral probiotic (e.g., Culturelle, Saccharomyces boulardii), at a dose has been stable for ≥2 weeks immediately prior to screening.
   May be receiving AZA, 6-MP, or MTX at a dose that has been stable for ≥12 weeks immediately prior to screening.
Must have received a colonoscopy within 2 years prior to screening or be willing to undergo a colonoscopy in lieu of a flexible sigmoidoscopy at screening. This colonoscopy must:
   Confirm disease extent, defined as 1) left-sided colitis (inflammation up to the splenic flexure), 2) extensive colitis (inflammation beyond the splenic flexure but not involving the entire colon), or 3) pancolitis (inflammation of the entire colon)
   Include removal of any adenomatous polyps
   Include surveillance to detect dysplasia per local standard of care for all patients with left-sided colitis of >12 years' duration or extensive colitis or pancolitis of >8 years' duration.

### Inclusion Criteria for Advanced Failures (Randomized and Open-Label Parts)

Advanced failure patients must meet the following criteria for study entry:
Inadequate response, loss of response to, or intolerance to up to 2 prior classes of approved advanced therapies, which include biologics and targeted small molecules, as defined by the following:
   Inadequate response is defined by persistent signs and symptoms of active disease despite at least one induction regimen of advanced therapy.
   Loss of response is defined by the recurrence of symptoms during maintenance dosing of advanced therapy following prior clinical benefit (discontinuation despite clinical benefit or discontinuation due to loss of access does not qualify).
   Intolerance is defined by history of adverse effect necessitating treatment discontinuation of advanced therapy (including, but not limited to infusion-related reaction, demyelination, congestive heart failure, infection).

### Inclusion Criteria for Conventional Failures (Randomized Part)

Conventional failure patients must meet the following criteria for study entry:
Inadequate response, loss of response to, or intolerance to prior conventional UC therapies (immunosuppressant and/or corticosteroid treatment)

Failure to only 5-ASA treatment is not sufficient.

Inadequate response to, loss of response to, or intolerance to prior immunosuppressant treatment (i.e., AZA, 6-MP, or MTX), is defined as one or more of the following:
Persistent signs and symptoms of active disease despite treatment with at least one 12-week regimen of AZA (≥1.5 mg/kg/day) or 6-MP
(≥0.75 mg/kg/day) and/or MTX (≥15 mg/week)
Persistent signs and symptoms of active disease despite a 6-thioguanine nucleotide level of ≥230 pmol/8×10⁸ RBCs (as measured by quantitative high-performance liquid chromatography or liquid chromatography/tandem mass spectrometry) during at least one 12-week regimen of oral AZA or 6-MP at a stable or increasing dose
History of intolerance to AZA, 6-MP, or MTX (including, but not limited to, nausea/vomiting, abdominal pain, pancreatitis, liver function test abnormalities, lymphopenia, TPMT genetic mutation, or infection)

Inadequate response, loss of response, or intolerance to corticosteroid treatment is defined as one or more of the following:
Steroid refractory: persistent symptoms of active disease despite treatment with at least one 4-week induction regimen that included ≥30 mg/day of oral prednisone (or equivalent) for at least 2 weeks or ≥30 mg/day of IV prednisone (or equivalent) for at least 1 week
Steroid dependent: two failed attempts to taper steroids below a dose equivalent to 10 mg/day of oral prednisone (or equivalent)
Steroid intolerant: history of intolerance to corticosteroids (including, but not limited to, Cushing's syndrome, osteopenia/osteoporosis, hyperglycemia, insomnia, or infection)

### Inclusion Criteria for Background Anti-TNF Therapy Cohort (Open-Label Part)

Patients in the background anti-TNF therapy cohort must meet the following criteria for study entry:
Must meet inclusion criteria for advanced failures (see above).
Currently receiving approved anti-TNF therapy that was initiated within 6 months of screening
Intends to continue approved anti-TNF therapy at stable doses during screening and the study treatment period
Ongoing treatment with anti-TNF therapy must be at stable doses for 8 weeks immediately prior to screening.
Adequate anti-TNF drug levels:
   Infliximab or infliximab biosimilar: trough concentration ≥5 μg/mL
   Adalimumab or adalimumab biosimilar: trough concentration >7.5 Mg/ml
   Golimumab or golimumab biosimilar: trough concentration ≥1 μg/mL
Despite adequate anti-TNF drug levels, inadequate response or loss of response to anti-TNF therapy, as defined by the following:
   Inadequate response is defined by persistent signs and symptoms of active disease despite at least one induction regimen of anti-TNF therapy.
   Loss of response is defined by recurrence of symptoms during maintenance dosing of anti-TNF therapy following prior clinical benefit (discontinuation despite clinical benefit does not qualify).

Allowed concomitant therapies include anti-inflammatoires such as 5-aminosalicylic acid (5-ASA) and oral corticosteroids, and immunosuppressants such as azathioprine (AZA), 6-mercaptopurine (6-MP), and methotrexate (MTX). For patients receiving an anti-TNF therapy according to the clinical protocol, examples of such anti-TNF therapies include infliximab, adalimumab, golimumab, or biosimilars. Prior treatment with a Janus kinase (JAK) inhibitor is exclusionary.

### Exclusion Criteria.

Exclusion criteria include:
Prior extensive colonic resection, subtotal or total colectomy, or planned surgery for UC
Past or present ileostomy or colostomy
Diagnosis of Crohn's disease or indeterminate colitis
Suspicion of ischemic colitis, radiation colitis, or microscopic colitis
Diagnosis of toxic megacolon within 12 months of initial screening visit
Past or present fistula or abdominal abscess
History or current evidence of unresected adenomatous colonic polyps or colonic mucosal dysplasia
Any stricture (stenosis) of the colon
Diagnosis or suspicion of primary sclerosing cholangitis
History of severe allergic reaction or anaphylactic reaction to a biologic agent
Previous treatment with vixarelimab
Previous treatment with tofacitinib, upadacitinib, or other systemic JAK inhibitors
Treatment of UC with approved biologic agents within 8 weeks or 5 half-lives prior to screening, whichever is longer, with the exception of the background anti-TNF therapy cohort
Treatment of UC with approved nonbiologic therapies other than those specifically listed as permitted medications within 30 days or 5 half-lives prior to screening, whichever is longer
Use of IV corticosteroids within 2 weeks prior to screening
Treatment with corticosteroid enemas or suppositories and/or topical (rectal) 5-ASA preparations within 2 weeks prior to screening
Apheresis (e.g., Adacolumn® apheresis) within 2 weeks prior to screening or intent to receive during the study
Chronic nonsteroidal anti-inflammatory drug (NSAID) use

### Method of Treatment Assignment and Blinding

This study consists of a three-arm randomized part and a single-arm open-label part. Patients in the randomized part will be randomly assigned to one of three treatment arms: the vixarelimab 720 mg Q2W arm, vixarelimab 720 mg Q4W arm, or placebo arm in a 1:1:1 ratio. During the optional ATE period, patients assigned to the vixarelimab 720 mg Q2W arm or the vixarelimab 720 mg Q4W arm during the induction period will continue on the randomized treatment assignment; patients treated with placebo during the induction period will receive vixarelimab 720 mg Q2W.

Patients in the open-label part will be assigned to the vixarelimab 720 mg Q2W arm; they will continue on this regimen during the optional ATE period.

### Clinical Outcome Assessments

Clinical outcome assessments of treatment benefit will be primarily collected through the Mayo Score, a composite endpoint incorporating both patient-reported outcome (PRO) and clinician-reported outcome (ClinRO) measures.

PRO data will be collected through use of the following instruments: Mayo Score (Stool Frequency and Rectal Bleeding items) and Ulcerative Colitis Patient-Reported Outcomes Signs and Symptoms (UC-PRO/SS). PRO instruments will capture each patient's direct experience with vixarelimab with and without anti-TNF background therapy.

ClinRO data will be collected through use of the Mayo Score (Physician's Global Assessment item). The Mayo Score (including mMS and pMS) will be assessed during the study at specified time points. The Mayo Score is a composite of four assessments, each having a scoring range of 0-3: stool frequency, rectal bleeding, endoscopy (score of 1 modified to exclude friability), and PGA. The Mayo Score is the sum of these assessment subscores and has a range of 0-12, with higher scores indicating more severe disease.

The mMS is a composite of three assessments from the Mayo Score, each having a scoring range of 0-3: stool frequency, rectal bleeding, and centrally read endoscopy. The mMS has a range of 0-9, with higher scores indicating more severe disease.

The pMS is a composite of three assessments from the Mayo Score, each having a scoring range of 0-3: stool frequency, rectal bleeding, and PGA. The pMS has a range of 0-9, with higher scores indicating more severe disease.

Stool frequency and rectal bleeding are components of the Mayo Score, mMS, and pMS.

At screening, the patient's normal number of stools, defined as the number of stools passed when a patient is in remission (i.e., not in flare), will be documented.

Patients are to record stool frequency and rectal bleeding daily in their electronic diary (e-diary) during screening and induction, beginning with the first screening visit, and at specified time points throughout the study. The average stool frequency and rectal bleeding scores from the daily e-diary entries within the 7-day period immediately prior to Day 1 (data from a minimum of 3 consecutive days or 4 nonconsecutive days) will be used as efficacy baseline and for inclusion.

Because the endoscopy and associated bowel preparation can interfere with the assessment of PROs, e-diary entries on days of bowel preparation, endoscopy, and the day after endoscopy will not be used to calculate any stool frequency or rectal bleeding scores.

Physician's Global Assessment (PGA) data will be collected through use of an electronic device/platform, or paper form. The PGA is a component of the Mayo Score and pMS. The PGA should reflect the clinician's assessment of the patient's current overall status, taking into account stool frequency and rectal bleeding scores, clinician endoscopy findings, patient-reported symptoms, clinician observations, physical examination findings, and other pertinent findings. If possible, the clinician who completes the PGA should not be the same clinician who assesses adverse events. If possible, the same clinician should complete the PGA at each time point.

Patients are to complete the UC-PRO/SS in the e-diary at specified time points throughout the study.

The UC-PRO/SS is a 9-item daily diary to quantify the effects of treatment on patient-reported signs and symptoms of UC. The UC-PRO/SS assesses the presence of UC symptoms and, in some cases, the severity or frequency of the symptoms (Higgins et al. 2017; Pulley et al. 2021). The UC-PRO/SS includes two domains, Bowel Signs and Symptoms (6 items) and Functional Symptoms (3 items), and yields two separate weekly average scores. Each scale is scored separately; there is no total score. Patients are instructed to complete the diary each evening, reflecting back on their experiences during the past 24 hours. The UC-PRO/SS takes approximately 3 minutes to complete. The UC-PRO/SS will be collected daily over at least a 7-day period before each specified study visit.

Because the endoscopy and associated bowel preparation can interfere with the assessment of PROs, e-diary entries on days of bowel preparation, endoscopy, and the day after endoscopy will not be used to calculate any stool frequency or rectal bleeding scores.

### Example 3. A Phase 2 Study to Evaluate Efficacy, Safety, and PK in Ulcerative Colitis (UC)

A Phase II, multicenter study was designed to evaluate the efficacy, safety, and pharmacokinetics of vixarelimab in patients with active moderate to severe UC, including 1) patients who have demonstrated inadequate response to, loss of response to, or intolerance to prior advanced therapy which includes biologics and targeted small molecules (advanced failures), and 2) patients who have demonstrated inadequate response to, loss of response to, or intolerance to prior conventional therapies (corticosteroids and/or immunosuppressants) but have not failed advanced therapy (conventional failures). Patients will be enrolled in either a randomized, parallel-group, double-bind, placebo controlled, dose-ranging part, or in an open-label active treatment part.

The study consists of screening period of up to 35 days, a 48-week treatment period, and a safety follow-up period for 10 weeks following the final dose of study treatment. The induction period (Weeks 0-12) will test the induction of clinical remission. After completion of the induction period, all patients, irrespective of clinical response or remission, will be eligible to continue study treatment during an active treatment extension (ATE) period (Weeks 12-48). During the ATE period, all patients will receive vixarelimab, including patients who received placebo during the induction period, and the durability of clinical response and remission will be explored.

TABLE 5
Clinical Trial Objectives and Corresponding Endpoints

| Primary Objective | Corresponding Endpoint |
|---|---|
| To evaluate the induction efficacy of | Clinical remission at Week 12, with clinical |
| vixarelimab compared with placebo | remission defined as mMS ^{a} of ≤ 2, |
| To evaluate the induction efficacy of | including: |
| vixarelimab compared with placebo in | Stool frequency subscore ≤ 1 |
| advanced failures | O Rectal bleeding subscore = 0 |
| | Endoscopy subscore ≤ 1 (score of 1 modified |
| | to exclude friability) ^{b} |
| To evaluate the induction efficacy of | Clinical response at Week 12, with clinical |
| vixarelimab compared with placebo | response defined as meeting both of the |
| To evaluate the induction efficacy of | following criteria: |
| vixarelimab compared with placebo in | Decrease from baseline in the mMS ^{a} of ≥ |
| advanced failures | 2 and ≥ 30% reduction from baseline |
| | Decrease in rectal bleeding subscore of ≥ |
| | 1 or absolute rectal bleeding subscore |
| | of ≤1 |
| | Endoscopic improvement at Week 12, with |
| | endoscopic improvement defined as a Mayo |
| | endoscopy subscore of ≤ 1 (score of 1 |
| | modified to exclude friability) ^{b} |
| | Endoscopic remission at Week 12, with |
| | endoscopic remission defined as a Mayo |
| | endoscopy subscore of 0 ^{b} |
| To evaluate the safety of vixarelimab | Incidence and severity of adverse events, |
| compared with placebo | with severity determined according to the |
| | DAIDS toxicity grading scale ^{c} |
| | Change from baseline in selected vital signs |
| | Change from baseline in selected clinical |
| | laboratory test results |
| To characterize the pharmacokinetics of | Serum concentration of vixarelimab at |
| vixarelimab | specified time points |
| To evaluate the immune response to | Prevalence of ADAs at baseline and incidence |
| vixarelimab | of ADAs during the study |
| To evaluate the induction efficacy of open- | Clinical remission at Week 12 |
| label vixarelimab | Clinical response at Week 12 |
| | Endoscopic improvement at Week 12 |
| | Endoscopic remission at Week 12 |
| To evaluate the long-term efficacy of | Sustained remission, defined as clinical |
| vixarelimab | remission at both Week 12 and Week 48 |
| | Clinical remission at Week 48 |
| | Clinical response at Week 48 |
| | Endoscopic improvement at Week 48 |
| | Endoscopic remission at Week 48 |
| | Change from baseline in UC bowel |
| | movement signs and symptoms at Week 12 |
| | and Week 48, as assessed by UC-PRO/SS |
| | score |
| | Improvement in UC bowel movement signs |
| | and symptoms at Week 12 and Week 48, as |
| | defined by the proportion of patients with |
| | a > 6-point decrease in the UC-PRO/SS |
| | bowel domain score |
| | Change from baseline in UC functional |
| | signs and symptoms at Week 12 and Week |
| | 48, as assessed by UC-PRO/SS score |
| | Improvement in UC functional symptoms at |
| | Week 12 and Week 48, as defined by the |
| | proportion of patients with a ≥ 2-point |
| | decrease in the UC-PRO/SS functional |
| | domain score |
| To evaluate potential relationships between | Relationship between PK parameters for |
| vixarelimab exposure and the efficacy and | vixarelimab and efficacy endpoints |
| safety of vixarelimab | Relationship between PK parameters for |
| | vixarelimab and safety endpoints |
| To evaluate potential relationships between | Relationship between selected covariates |
| selected covariates and exposure to | and PK parameters for vixarelimab |
| vixarelimab | |
| To evaluate potential effects of ADAs | Relationship between ADA status and |
| | efficacy, safety, or PK endpoints |
| To identify and/or evaluate biomarkers that | Relationship between biomarkers in blood, |
| are predictive of response to vixarelimab | serum, stool, and colonic tissue, and |
| (i.e., predictive biomarkers), are associated | efficacy, safety, PK, immunogenicity, or |
| with progression to a more severe disease | other biomarker endpoints |
| state (i.e., prognostic biomarkers), are | |
| associated with acquired resistance to | |
| vixarelimab, are associated with susceptibility | |
| to developing adverse events or can lead to | |
| improved adverse event monitoring or | |
| investigation (i.e., safety biomarkers), can | |
| provide evidence of vixarelimab activity or | |
| disease improvement (i.e., PD biomarkers), | |
| can serve as a useful surrogate endpoint, or | |
| can increase the knowledge and | |
| understanding of disease biology and drug | |
| safety or pharmacokinetics | |
| To evaluate the use of automated assessment | Automated assessment of disease severity |
| of endoscopic videos to infer the efficacy of | Automated spatial assessment of mucosal |
| vixarelimab | features |

ADA = anti-drug antibody; DAIDS = Division of AIDS; mMS = modified Mayo Score; PD = pharmacodynamic; PK = pharmacokinetic; UC = ulcerative colitis; UC-PRO/SS = Ulcerative Colitis Patient-Reported Outcomes Signs and Symptoms.
^{a} Modified Mayo Score is the composite of three Mayo Score assessments: stool frequency, rectal bleeding, and centrally read endoscopy (see FIG. 7).
^{b} Endoscopy scores will be based on interpretation by a blinded central reader.
^{c} Adverse events will be graded according to the DAIDS Table for Grading the Severity of Adult and Pediatric Adverse Events (HHS 2017), with slight modifications for clarity and for alignment with internal practices.

The eligibility criteria for this study define patients with active moderate to severe UC with an mMS of 5-9 and endoscopic score of at least 2, consistent with regulatory guidance (U.S. Food and Drug Administration [FDA] 2022), who may benefit from the anticipated effects of vixarelimab. All patients considered for participation will have a diagnosis of moderate to severe UC established at least 3 months prior to screening, with active disease confirmed by clinical and endoscopic evidence during screening. Patients must have demonstrated an inadequate response, loss of response, or intolerance to prior conventional UC therapies and/or to up to 2 prior classes of approved advanced therapies (as defined below in “Inclusion Criteria for Advanced Failures”).

Patients will undergo an endoscopy with biopsy and a full Mayo Score assessment at screening (baseline), Week 12, and Week 48. Efficacy will be assessed using the modified Mayo Score (mMS), with the Mayo endoscopic subscore calculated on the basis of centrally read endoscopy. The mMS and partial Mayo Score (pMS) will be derived from the Mayo Score.

Patients who complete the treatment period (induction and optional ATE) will enter the safety follow-up period and undergo assessments at Week 52 and Week 56. Patients who discontinue study treatment without entering the optional ATE period will enter the safety follow-up period and undergo assessments at 6 weeks and 10 weeks following the final dose of study treatment. Patients who discontinue study treatment prematurely should return to the clinic for a treatment discontinuation visit within 14 days of the event and will then enter the safety

The total duration of study participation for each patient is expected to be approximately 25 weeks for patients who complete the induction treatment period only, and 61 weeks for patients who complete the optional ATE.

### Randomized Treatment Part

Approximately 210 patients, including advanced failures and conventional failures as defined below, will be enrolled in the randomized, double-blind treatment part of the study. The number of conventional failures will not exceed 40% of enrolled patients in the randomized part. Randomization will be stratified by previous advanced or conventional therapy failure and baseline modified Mayo Score (mMS).

Eligible patients will be randomized in a 1:1:1 ratio to one of the following treatment arms for the induction period (Weeks 0-12):
Vixarelimab 720 mg Q2W (720 mg at Weeks 0, 1, 2, and Q2W thereafter).
Vixarelimab 720 mg Q4W arm (720 mg at Weeks 0, 1, 4, and Q4W thereafter; placebo at Week 2 and Q4W thereafter).
Placebo Q2W arm (placebo at Weeks 0, 1, 2, and Q2W thereafter).

All patients who complete the induction period can receive SC injections of vixarelimab 720 mg during the optional ATE period (Weeks 12-48). Patients assigned to the vixarelimab 720 mg Q2W arm or vixarelimab 720 mg Q4W arm during the induction period will continue on their randomized treatment assignment during the optional ATE period:
Patients in the vixarelimab 720 mg Q2W arm will receive vixarelimab 720 mg Q2W.
Patients in the vixarelimab Q4W arm will receive vixarelimab 720 mg Q4W (starting at Week 12 and Q4W thereafter) and placebo Q4W (starting at Week 14 and Q4W thereafter).
Patients in the placebo arm during the induction period will receive vixarelimab 720 mg Q2W during the optional ATE period.

### Open-Label Treatment Part

Up to 40 advanced failure patients will be enrolled in a separate open-label treatment part of the study. This open-label treatment part may be initiated after approximately 6 months of global enrollment in the randomized part of the study. If enrollment is significantly below projected expectations, the open-label treatment will be open to enable an initial proof of activity within a reasonable timeframe and advanced failure patients will be enrolled in the open-label treatment part until 40 advanced failure patients are enrolled, after which enrollment of advanced failure patients will resume in the randomized treatment part of the study.

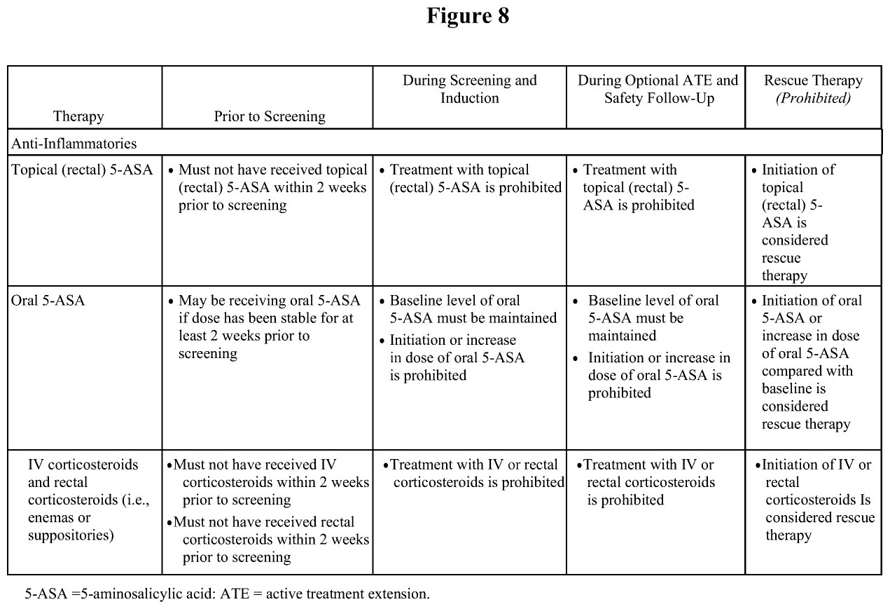

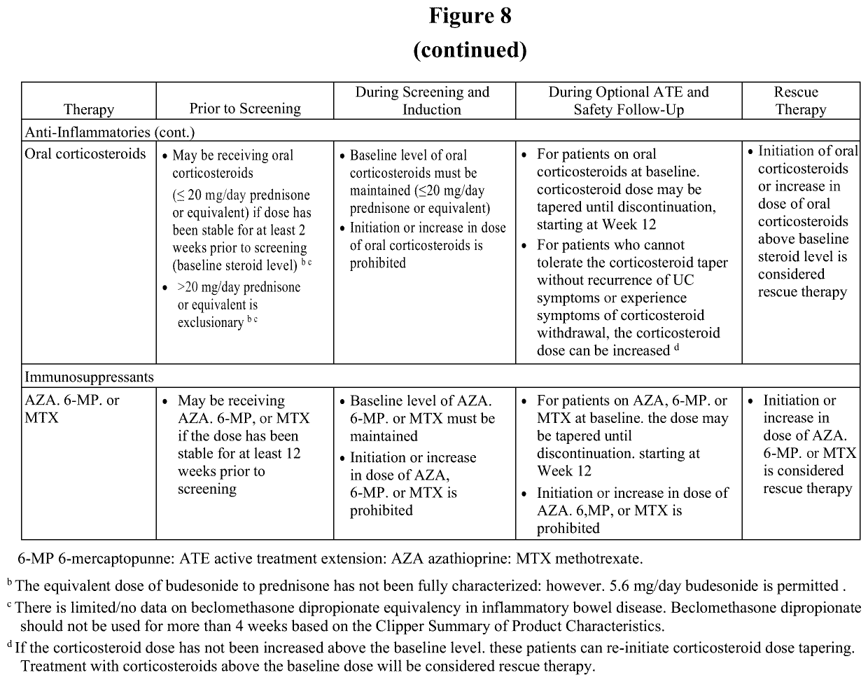

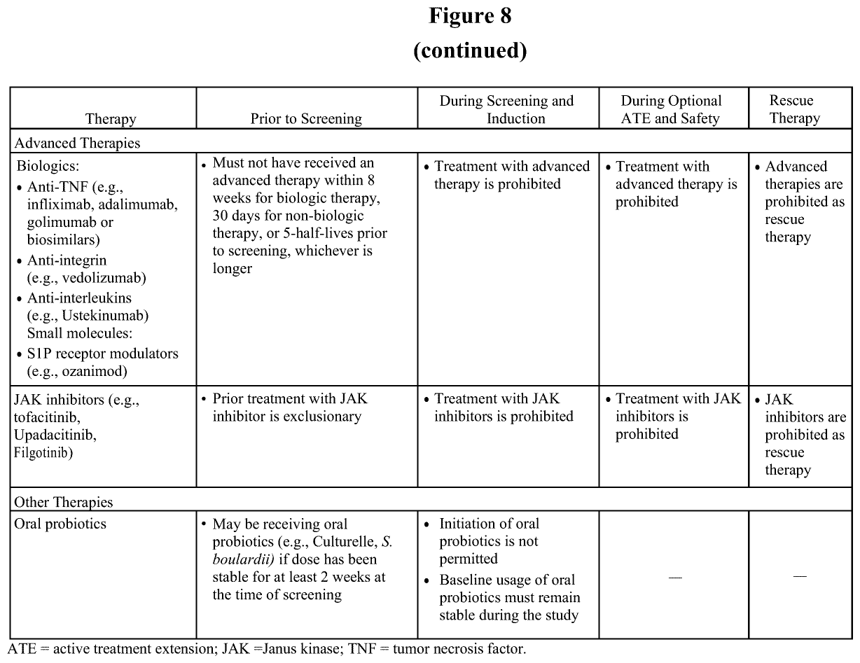

All patients in the open-label treatment part will be assigned to the vixarelimab 720 mg Q2W arm to receive SC injections of vixarelimab 720 mg at Week 0, 1, 2, and Q2W thereafter during the induction period (Weeks 0-12) and continue with 720 mg Q2W during the optional ATE period (Weeks 12-48), followed by follow-up visits approximately 6 weeks and 10 weeks after the final dose; these patients will undergo the same schedule of assessments as patients in the randomized treatment part. Restrictions regarding treatment with concomitant therapy are outlined in FIG. 8.

The total duration of study participation for each patient is expected to be approximately 25 weeks for patients who complete the induction treatment period only, and 61 weeks for patients who complete the optional ATE.

### General Inclusion Criteria for All Patients

Inclusion criteria for study entry include:
≥18 years of age at time of signing Informed Consent Form
Ability to comply with the study protocol
Diagnosis of UC established at least 3 months prior to screening, confirmed by clinical and endoscopic evidence during screening. The diagnosis of UC should be corroborated by histopathology and documented by a histopathology report. A histopathologic examination should be performed at screening if no prior report is readily available.
Evidence of UC extending a minimum of 15 cm from the anal verge as determined by baseline endoscopy (flexible sigmoidoscopy or colonoscopy) performed during screening
Moderately to severely active UC, defined as a mMS of 5-9 with an endoscopic subscore of ≥2 (as determined by the central reading procedure described in Section 4.5.6) during the screening period, despite concurrent treatment
Any permitted background UC therapy must be at stable doses, as outlined below:
   May be receiving oral 5-ASA compounds at a dose that has been stable for ≥2 weeks immediately prior to screening.
   May be receiving oral corticosteroid therapy at a dose of ≤20 mg/day of prednisone (or equivalent) that has been stable for >2 weeks immediately prior to screening.
   May be receiving oral probiotic (e.g., Culturelle, Saccharomyces boulardii), at a dose has been stable for >2 weeks immediately prior to screening.
   May be receiving AZA, 6-MP, or MTX at a dose that has been stable for ≥12 weeks immediately prior to screening.
Must have received a colonoscopy within 2 years prior to screening or be willing to undergo a colonoscopy in lieu of a flexible sigmoidoscopy at screening. This colonoscopy must:
   Confirm disease extent, defined as 1) left-sided colitis (inflammation up to the splenic flexure), 2) extensive colitis (inflammation beyond the splenic flexure but not involving the entire colon), or 3) pancolitis (inflammation of the entire colon)
   Include removal of any adenomatous polyps
   Include surveillance to detect dysplasia per local standard of care for all patients with left-sided colitis of >12 years' duration or extensive colitis or pancolitis of >8 years' duration.

### Inclusion Criteria for Advanced Failures (Randomized and Open-Label Parts)

Advanced failure patients must meet the following criteria for study entry:
Inadequate response, loss of response to, or intolerance to up to 2 prior classes of approved advanced therapies, which include biologics and targeted small molecules, as defined by the following:
   Inadequate response is defined by persistent signs and symptoms of active disease despite at least one induction regimen of advanced therapy.
   Loss of response is defined by the recurrence of symptoms during maintenance dosing of advanced therapy following prior clinical benefit (discontinuation despite clinical benefit or discontinuation due to loss of access does not qualify).
   Intolerance is defined by history of adverse effect necessitating treatment discontinuation of advanced therapy (including, but not limited to infusion-related reaction, demyelination, congestive heart failure, infection).

### Inclusion Criteria for Conventional Failures (Randomized Part)

Conventional failure patients must meet the following criteria for study entry:
Inadequate response, loss of response to, or intolerance to prior conventional UC therapies (immunosuppressant and/or corticosteroid treatment)

Failure to only 5-ASA treatment is not sufficient.

Inadequate response to, loss of response to, or intolerance to prior immunosuppressant treatment (i.e., AZA, 6-MP, or MTX), is defined as one or more of the following:
Persistent signs and symptoms of active disease despite treatment with at least one 12-week regimen of AZA (≥1.5 mg/kg/day) or 6-MP
(≥0.75 mg/kg/day) and/or MTX (≥15 mg/week)
Persistent signs and symptoms of active disease despite a 6-thioguanine nucleotide level of ≥230 pmol/8×10⁸ RBCs (as measured by quantitative high-performance liquid chromatography or liquid chromatography/tandem mass spectrometry) during at least one 12-week regimen of oral AZA or 6-MP at a stable or increasing dose
History of intolerance to AZA, 6-MP, or MTX (including, but not limited to, nausea/vomiting, abdominal pain, pancreatitis, liver function test abnormalities, lymphopenia, TPMT genetic mutation, or infection)

Inadequate response, loss of response, or intolerance to corticosteroid treatment is defined as one or more of the following:
Steroid refractory: persistent symptoms of active disease despite treatment with at least one 4-week induction regimen that included ≥30 mg/day of oral prednisone (or equivalent) for at least 2 weeks or ≥30 mg/day of IV prednisone (or equivalent) for at least 1 week
Steroid dependent: two failed attempts to taper steroids below a dose equivalent to 10 mg/day of oral prednisone (or equivalent)
Steroid intolerant: history of intolerance to corticosteroids (including, but not limited to, Cushing's syndrome, osteopenia/osteoporosis, hyperglycemia, insomnia, or infection)

### Exclusion Criteria.

Exclusion criteria include:
Prior extensive colonic resection, subtotal or total colectomy, or planned surgery for UC
Past or present ileostomy or colostomy
Diagnosis of Crohn's disease or indeterminate colitis
Suspicion of ischemic colitis, radiation colitis, or microscopic colitis
Diagnosis of toxic megacolon within 12 months of initial screening visit
Past or present fistula or abdominal abscess
History or current evidence of unresected adenomatous colonic polyps or colonic mucosal dysplasia
Any stricture (stenosis) of the colon
Diagnosis or suspicion of primary sclerosing cholangitis
History of known or suspected allergic reaction or anaphylactic reaction to a biologic agent, including vixarelimab or its excipients
Previous treatment with vixarelimab
Previous treatment with tofacitinib, upadacitinib, or other systemic JAK inhibitors
Treatment of UC with approved biologic agents within 8 weeks or 5 half-lives prior to screening, whichever is longer
Treatment of UC with approved nonbiologic therapies other than those specifically listed as permitted medications within 30 days or 5 half-lives prior to screening, whichever is longer
Use of IV corticosteroids within 2 weeks prior to screening
Treatment with corticosteroid enemas or suppositories and/or topical (rectal) 5-ASA preparations within 2 weeks prior to screening
Apheresis (e.g., Adacolumn® apheresis) within 2 weeks prior to screening or intent to receive during the study
Chronic nonsteroidal anti-inflammatory drug (NSAID) use

### Method of Treatment Assignment and Blinding

This study consists of a three-arm randomized part and a single-arm open-label part. Patients in the randomized part will be randomly assigned to one of three treatment arms: the vixarelimab 720 mg Q2W arm, vixarelimab 720 mg Q4W arm, or placebo arm in a 1:1:1 ratio. During the optional ATE period, patients assigned to the vixarelimab 720 mg Q2W arm or the vixarelimab 720 mg Q4W arm during the induction period will continue on the randomized treatment assignment; patients treated with placebo during the induction period will receive vixarelimab 720 mg Q2W.

Patients in the open-label part will be assigned to the vixarelimab 720 mg Q2W arm; they will continue on this regimen during the optional ATE period.

If the open-label part is initiated, advanced failure patients will be enrolled in the open-label part until enrollment is fulfilled, after which advanced failure patients will continue to be enrolled in the randomized part.

### Clinical Outcome Assessments

Clinical outcome assessments of treatment benefit will be primarily collected through the Mayo Score, a composite endpoint incorporating both patient-reported outcome (PRO) and clinician-reported outcome (ClinRO) measures.

PRO data will be collected through use of the following instruments: Mayo Score (Stool Frequency and Rectal Bleeding items) and Ulcerative Colitis Patient-Reported Outcomes Signs and Symptoms (UC-PRO/SS). PRO instruments will capture each patient's direct experience with vixarelimab.

ClinRO data will be collected through use of the Mayo Score (Physician's Global Assessment item). The Mayo Score (including mMS and pMS) will be assessed during the study at specified time points. The Mayo Score is a composite of four assessments, each having a scoring range of 0-3: stool frequency, rectal bleeding, endoscopy (score of 1 modified to exclude friability), and PGA. The Mayo Score is the sum of these assessment subscores and has a range of 0-12, with higher scores indicating more severe disease.

The mMS is a composite of three assessments from the Mayo Score, each having a scoring range of 0-3: stool frequency, rectal bleeding, and centrally read endoscopy. The mMS has a range of 0-9, with higher scores indicating more severe disease.

The pMS is a composite of three assessments from the Mayo Score, each having a scoring range of 0-3: stool frequency, rectal bleeding, and PGA. The pMS has a range of 0-9, with higher scores indicating more severe disease.

Stool frequency and rectal bleeding are components of the Mayo Score, mMS, and pMS.

At screening, the patient's normal number of stools, defined as the number of stools passed when a patient is in remission (i.e., not in flare), will be documented.

Patients are to record stool frequency and rectal bleeding daily in their electronic diary (e-diary) during screening and induction, beginning with the first screening visit, and at specified time points throughout the study. The average stool frequency and rectal bleeding scores from the daily e-diary entries within the 7-day period immediately prior to Day 1 (data from a minimum of 3 consecutive days or 4 nonconsecutive days) will be used as efficacy baseline and for inclusion.

Because the endoscopy and associated bowel preparation can interfere with the assessment of PROs, e-diary entries on days of bowel preparation, endoscopy, and the day after endoscopy will not be used to calculate any stool frequency or rectal bleeding scores.

Physician's Global Assessment (PGA) data will be collected through use of an electronic device/platform, or paper form. The PGA is a component of the Mayo Score and pMS. The PGA should reflect the clinician's assessment of the patient's current overall status, taking into account stool frequency and rectal bleeding scores, clinician endoscopy findings, patient-reported symptoms, clinician observations, physical examination findings, and other pertinent findings. If possible, the clinician who completes the PGA should not be the same clinician who assesses adverse events. If possible, the same clinician should complete the PGA at each time point.

Patients are to complete the UC-PRO/SS in the e-diary at specified time points throughout the study.

The UC-PRO/SS is a 9-item daily diary to quantify the effects of treatment on patient-reported signs and symptoms of UC. The UC-PRO/SS assesses the presence of UC symptoms and, in some cases, the severity or frequency of the symptoms (Higgins et al. 2017; Pulley et al. 2021). The UC-PRO/SS includes two domains, Bowel Signs and Symptoms (6 items) and Functional Symptoms (3 items) and yields two separate weekly average scores. Each scale is scored separately; there is no total score. Patients are instructed to complete the diary each evening, reflecting back on their experiences during the past 24 hours. The UC-PRO/SS takes approximately 3 minutes to complete. The UC-PRO/SS will be collected daily over at least a 7-day period before each specified study visit.

Because the endoscopy and associated bowel preparation can interfere with the assessment of PROs, e-diary entries on days of bowel preparation, endoscopy, and the day after endoscopy will not be used to calculate any stool frequency or rectal bleeding scores.

Although the foregoing disclosure has been described in some detail by way of illustration and example for purposes of clarity of understanding, the descriptions and examples should not be construed as limiting the scope of the disclosure. The disclosures of all patent and scientific literature cited herein are expressly incorporated in their entirety by reference.

1. A method for treating an inflammatory bowel disease (IBD) comprising administering to a human subject in need thereof a therapeutically effective amount of vixarelimab.

2. The method of claim 1, wherein the administering comprises administering the therapeutically effective dose subcutaneously.

3. The method of claim 1, wherein the IBD is ulcerative colitis (UC).

4. The method of claim 3, wherein the UC is active moderate to severe UC.

5.-7. (canceled)

8. The method of claim 2, wherein the therapeutically effective dose is about 360 mg to 720 mg of the anti-OSMRβ antibody.

9. The method of claim 2, wherein the therapeutically effective dose is about 500 mg to 720 mg of the anti-OSMRβ antibody.

10. The method of claim 2, wherein the therapeutically effective dose is about 720 mg of the anti-OSMRβ antibody.

11. The method of claim 2, wherein the therapeutically effective dose is administered once every week, once every 2 weeks, once every 3 weeks, once every 4 weeks, or once every month.

12. The method of claim 2, wherein the administering comprises administering the therapeutically effective dose once per week for 1, 2, 3, 4, or 5 weeks followed by administering the therapeutically effective dose once every 2 weeks, once every 3 weeks, once every 4 weeks, or once every month.

13. The method of claim 2, wherein the administering comprises administering the therapeutically effective dose once per week for 3 weeks followed by administering the therapeutically effective dose once every 3 weeks.

14. The method of claim 2, wherein the administering comprises administering the therapeutically effective dose once per week for 3 weeks followed by administering the therapeutically effective dose once every 2 weeks.

15. The method of claim 2, wherein the administering comprises administering the therapeutically effective dose once per week for 3 weeks followed by administering the therapeutically effective dose once every 4 weeks.

16. The method of claim 2, wherein the administering comprises administering the therapeutically effective dose once every 2 weeks.

17.-21. (canceled)

22. The method of claim 2, wherein the treatment with a therapeutically effective dose of anti-OSMRβ for at least 10, 11, or 12 weeks results in a clinical response in the subject.

23.-33. (canceled)

34. A method for treating a human subject having active moderate-to-severe ulcerative colitis comprising subcutaneously administering to the human subject a therapeutically effective dose of vixarelimab.

35.-36. (canceled)

37. The method of claim 34, wherein the therapeutically effective dose is about 720 mg administered at weeks 0, 1, 2, and every 2 weeks (Q2W) thereafter.

38. The method of claim 34, wherein the therapeutically effective dose is about 720 mg administered at weeks 0, 1, 4, and every 4 weeks (Q4W) thereafter.

39. The method of claim 34, wherein the therapeutically effective dose is about 720 mg administered at Q2W.

40. The method of claim 34, wherein the therapeutically effective dose is administered for at least 12 weeks.

41. The method, of claim 40, wherein the treatment results in a clinical response.

42. The method, of claim 40, wherein the treatment results in clinical remission.

43. The method of claim 34, wherein the therapeutically effective dose is administered for 48 weeks.

44. The method, of claim 41, wherein the therapeutically effective dose is administered for 48 weeks, and the clinical response is sustained at 48 weeks.

45. The method, of claim 42, wherein the therapeutically effective dose is administered for 48 weeks, and the clinical remission is sustained at 48 weeks.

46. The method of claim 41, wherein the clinical response comprises a decrease from baseline in the Modified Mayo Score (mMS) of ≥2 and ≥30% reduction from baseline, a decrease in rectal bleeding subscore of ≥1 and/or an absolute rectal bleeding subscore of ≤1.

47. The method of claim 41, of claim 41, wherein the clinical response comprises endoscopic improvement at week 12.

48. The method of claim 41, wherein the clinical response comprises endoscopic remission at week 12.

49. The method, of claim 47, wherein the therapeutically effective dose is administered for at least 48 weeks.

50. The method, of claim 49, wherein the endoscopic improvement is sustained at week 48.

51. The method, of claim 48, wherein the therapeutically effective dose is administered for at least 48 weeks.

52. The method, of claim 51, wherein the endoscopic remission is sustained at week 48.

53. The method of claim 34, wherein the treatment results in improved signs or improved symptoms.

54. The method, of claim 53, wherein the improved signs or improved symptoms comprise an improvement in ulcerative colitis bowel movement signs and symptoms defined as the proportion of subjects with a ≥6-point decrease in the UC-PRO/SS bowel domain score.

55. The method, of claim 53, wherein the improved signs or improved symptoms comprise a change from baseline in UC functional signs and symptoms as assessed by IC-PRO/SS score

56. The method, the vixarelimab for use or the use of claim 53, wherein the improved signs or improved symptoms comprise an improvement in ulcerative colitis functional symptoms as defined by proportion of subjects with a >2-point increase in the UC-PRO/SS functional domain score.

57. The method of claim 53, wherein the improved signs or improved symptoms are assessed at week 12 and week 48.

58. The method of claim 34, wherein the treatment with a therapeutically effective dose of vixarelimab for at least 48 weeks results in a sustained clinical or endoscopic remission.

59. The method of claim 34, wherein the subject has (1) inadequate response to, loss of response to, or intolerance to up to two prior classes of approved ulcerative colitis advanced therapies; and/or (2) inadequate response to, loss of response to, or intolerance to prior conventional ulcerative colitis therapies; and/or (3) inadequate response to, loss of response to, or intolerance to prior immunosuppressant treatment.

60. The method of claim 59, wherein the subject has inadequate response to, loss of response to, or intolerance to up to two prior classes of approved ulcerative colitis advanced therapies.

61. The method of claim 59, wherein the subject has inadequate response to, loss of response to, or intolerance to prior conventional ulcerative colitis therapies.

62. The method of claim 59, wherein the subject has inadequate response to, loss of response to, or intolerance to prior immunosuppressant treatment.

63. The method of claim 34, wherein the vixarelimab is administered to the subject in combination with a second therapeutic agent.

64. The method, of claim 63, wherein the second therapeutic agent is an anti-TNF antibody.

65. The method, of claim 64, wherein the anti-TNF antibody is infliximab, adalimumab, golimumab, or a biosimilar thereof.

66. The method, of claim 63, wherein the second therapeutic agent is an anti-inflammatory.

67. The method, of claim 66, wherein the anti-inflammatory is 5-aminosalicylic acid or a corticosteroid.

68. The method, of claim 63, wherein the second therapeutic agent is an immunosuppressant.

69. The method, of claim 68, wherein the immunosuppressant is azathioprine, methotrexate, or 6-mercaptopurine.


SEQUENCE LISTING (4 sequences)

SEQ ID NO: 1 - protein, 452 aa, Synthetic construct
QVQLVQSGAEVKKPGASVKVSCKASGYTFTSYEINWVRQATGQGLEWMGWMNPNSGYTGYAQKFQGRVTMTRDTSISTAYMEMSSLRSEDTAVYYCARDIVAANTDYYFYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 216 aa, Synthetic construct
QSVLTQPPSASGTPGQRVTISCSGSNSNIGSNTVNWYHQLPGTAPKLLIYNINKRPSGVPDRFSGSKSGSSASLAISGLQSEDEADYYCSTWDDSLDGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 126 aa, Synthetic construct
QVQLVQSGAEVKKPGASVKVSCKASGYTFTSYEINWVRQATGQGLEWMGWMNPNSGYTGYAQKFQGRVTMTRDTSISTAYMEMSSLRSEDTAVYYCARDIVAANTDYYFYYGMDVWGQGTTVTVSS

SEQ ID NO: 4 - protein, 111 aa, Synthetic construct
QSVLTQPPSASGTPGQRVTISCSGSNSNIGSNTVNWYHQLPGTAPKLLIYNINKRPSGVPDRFSGSKSGSSASLAISGLQSEDEADYYCSTWDDSLDGVVFGGGTKLTVLG